(19) United States
(12) Patent Application Publication
(10) Pub. No.: US 2008/0131366 A1
(43) Pub. Date: Jun. 5, 2008

(54) Folate Receptor Gene Modulation For Cancer Diagnosis And Therapy

(76) Inventor: Manohar Ratnam, Toledo, OH (US)

Correspondence Address: Patrick P. Pacella;Emch Schaffer Schaub & Porcello Co., PO Box 916, Toledo, OH, 43697-0916, US

(21) Appl. No.: 10/549,662

(22) PCT Filed: Mar. 16, 2004
(86) PCT No.: PCT/US04/08129

§ 371 Date: Mar. 23, 2007

Related U.S. Application Data
(60) Provisional application No. 60/455,705, filed on Mar. 17, 2003.

(51) Int. Cl. A61K 31/137; A61K 31/56; A61K 31/165; A61K 9/127; A61K 51/00; C12N 5/00; A61P 35/04

(52) U.S. Cl. 424/1.65; 514/648; 514/171; 514/619; 424/450; 435/375; 435/325

(57) ABSTRACT

A method of diagnosing and treating a patient with or suspected of having at least one solid tumor that produces folate receptor type α includes administering a biologically effective amount of at least one folate receptor α inducer, specifically steroid receptor ligands, with or without aid from at least one histone deacetylase inhibitor, to increase the level of the folate receptor α on the plasma membrane of cancer cells as well as the folate receptor α in at least one type of body fluid, including, for example, serum, ascites and cerebrospinal fluid.

This invention was made, at least in part, with government support under National Institutes of Health R01 Grants CA80183 and CA 92890. The U.S. government has certain rights in the invention.

### FIELD OF THE INVENTION

This invention relates to methods of diagnosing and treating patients with many types of cancer using agents which up-regulate expression of folate receptor α (FR-α) to enable the utilization of FR-α in diagnostic and therapeutic applications.

### BACKGROUND OF THE INVENTION

About 16 million Americans have been diagnosed with cancer since 1990. Each year about 1.3 million new cancer cases are expected to be diagnosed, and about 555,500 Americans are expected to die of cancer; this amounts to an average of greater than 1,500 cancer deaths a day. Cancer is the second leading cause of death in the US, surpassed only by heart disease. In the US, 1 of every 4 deaths is from cancer. The National Institutes of Health estimate overall costs of cancer in the year 2001 at $156.7 billion, with a total direct medical cost of $56.4 billion. Traditionally, the most effective method of treating solid tumors such as cancers of the genital system, which have a high incidence in industrialized countries, has been surgery, radiation therapy, and chemotherapy followed by anticancer drugs as adjuvant therapy. Unfortunately, 90% of cancers of the genital system, including ovarian cancer, uterine endometrial cancer and breast cancer are diagnosed at a late stage, when there is extensive metastasis. For these cancers, cytotoxic anti-cancer drugs have become the first line of treatment. These treatments are predominantly systemic and highly toxic to normal tissues.

Early detection will significantly improve the survival rate following treatment. For many cancers, there is also a need for an effective means of monitoring patients after initial successful treatment for recurrence or spread of the cancer in order to enable timely follow up treatment. Proteins called tumor markers, that are either selectively expressed in cancerous tissues and/or are released into circulation by the tumor cells, can serve to detect cancers early and to monitor their recurrence after treatment. One example of a tumor marker is the folate receptor, which is produced by tumor cells in major gynecological and other cancers.

Tumor targeted drug delivery systems can potentially greatly improve the outcome of cancer treatment. Reformulated versions of older as well as new treatments including immunological therapies are being developed. These versions incorporate specific tumor targeting, which will reduce the toxicity to normal tissues/cells and will dramatically increase the effectiveness of cancer therapeutics.

Many of the above experimental treatments have worked well in animal models; however, in contrast to the model tumors grown in animals, human cancers are notoriously variable in the key characteristics utilized by targeted therapeutics. To be specific, all parts of the same tumor or the same type of tumor from different individuals will not produce an adequate amount of a marker protein (e.g. folate receptor) that earmarks the tumor for detection or for targeted therapeutics. This limitation also confounds the ability to use the folate receptor in cancer diagnosis assays of samples of body fluids and in imaging tumors.

Redressing the critical problem of the variability and low levels of expression of the folate receptor in tumors will enable more effective application of a broad range of promising folate receptor-mediated cancer diagnostics and therapeutics in the clinic.

### SUMMARY OF THE INVENTION

In the limited number of normal tissues in which the folate receptor (FR) type α is expressed, the receptor is restricted to apical (luminal) surfaces, where it is inaccessible via the circulation. As a consequence, FR-α is regarded as a promising target, in major subtypes of ovarian, uterine, testicular and other cancers, for the selective delivery of a wide variety of diagnostic and therapeutic agents through the blood stream and as a serum marker for these cancers. Even though a large and growing body of evidence from pre-clinical and clinical studies supports the feasibility of developing such diagnostics/therapies, translating the success obtained in animal models to human cancer is confounded by variability and heterogeneity in the expression levels of FR-α in the tumors. This invention circumvents the problem of low expression levels of FR-α in individual tumors and parts thereof.

In one aspect, the present invention relates to the use of agonists or antagonists of steroid receptors (collectively called steroid receptor ligands). These agents substantially activate the FR-α gene in steroid receptor-positive tumors by acting through the steroid receptors. This results in a striking increase in the expression of FR-α in the treated cells. The present examples show that the FR-α gene can be activated through steroid receptors, which includes a family of closely related proteins, namely the estrogen receptor (ER), the progesterone receptor (PR), the glucocorticoid receptor (GR) and the androgen receptor (AR), greatly expanding the repertoire of FR-α⁺ tumors in which the receptor expression may be optimized for effective diagnostic/therapeutic applications. The detailed mechanistic examples presented herein show that human tumors that express ER, PR, GR or AR and in which the FR-α gene is already turned on will respond to brief (a few days) treatment with steroid receptor ligands by increasing the production of FR-α by an amount between one and two orders of magnitude. Antagonists of ER (such as tamoxifen) and agonists of PR, GR and AR (such as progesterone, dexamethasone and testosterone and their synthetic analogs) increase FR-α expression selectively in the FR-α⁺ tumor cells. Further, the present examples also show that none of these agents may be expected to induce FR expression in tissues that are ordinarily FR-negative; thus they are believed not to alter the pattern of FR expression in various tissues and are believed to retain the tumor selectivity of the FR-targeted diagnostic and therapeutic agents. Furthermore, the examples herein show that trichostatin A, a histone deacetylase inhibitor, potentiates the above effects of steroid receptor antagonists or agonists. Trichostatin A will not by itself substantially induce FR-α expression but will enable steroid receptor ligands to act more effectively. Therefore, while not wishing to be bound by theory, it is the inventor's belief that many histone deacetylase inhibitors, which have been tolerated in clinical trials as antitumor agents, are also useful in combination with steroid receptor agonists/antagonists to aid in FR induction in tumors cells.

In another aspect, the present invention relates to the use of molecules that bind steroid receptors, individually or in combination, to substantially increase folate receptor (FR) type α expression in a broad range of tumors in a manner selective to FR-α-positive tissues. This manipulation of tumor tissues using molecules that bind to steroid receptors together with or without histone deacetylase inhibitors, enhances the sensitivity of whole body imaging and the therapeutic efficacy of FR-targeted agents in the treatment of major types of cancer and also increases the level of FR-α in circulation, enabling their application as serum markers for diagnostic screening as well as monitoring of cancers during and after treatment.

In another aspect, the present invention provides methods of diagnosing and treating patients with or suspected of having various solid tumors that produce folate receptor type α. The method comprises administering biologically effective amounts of agents (referred to hereinafter as a “FR-α inducers”) that increase the level of folate-receptor a (FR-α) on the plasma membrane of cancer cells as well as FR-α in body fluids (serum, ascites and cerebrospinal fluid) followed by (i) a diagnostic assay using samples of body fluids, (ii) diagnostic tumor imaging using imaging agents directed at FR-α or (iii) administering a therapeutic agent that targets FR-α expressed by the tumor cells. In one embodiment, the FR-α inducers are agonists or antagonists of the steroid hormone receptors. Examples of such agents are tamoxifen, progestin, androgens and dexamethasone.

In another embodiment, a histone deactylase inhibitor is administered to the patient together with one or more steroid receptor agonist or antagonists. Examples of suitable histone deactylase inhibitors are trichostatin A (TSA) and suberoylanilide hydroxamic acid (SAHA), valproic acid, butyrates and depsipeptide.

In certain embodiments, the tools for assaying folate receptor in serum and ascites are fluorescent conjugates of folate or antibodies specific for the folate receptor. Also, in certain embodiments, the tools for therapeutic targeting of the folate receptor include any of a wide range of agents that have been described in the literature, including but not restricted to folate-coated liposomal drugs, folate conjugated nanoparticle drug delivery systems, antifolate drugs, folate conjugated radiopharmaceuticals or cytotoxics or folate receptor-targeted immunotherapeutics.

Other features and advantages of the invention will be apparent from the detailed description and from the claims. Although materials and methods similar or equivalent to those described herein can be used in the practice or testing of the invention, the preferred materials and methods are described below.

### BRIEF DESCRIPTION OF THE DRAWINGS

FIGS. 1A, 1B and 1C are graphs showing a promoter-specific repression of FR-α transcription by ER in model cell lines. (FIG. 1A) HeLa and IGROV-1 cells (10⁶) were co-transfected with the FR-α promoter-luciferase construct (400 ng) and either an ER expression plasmid (25 ng) or with the plasmid vector alone (25 ng) in the presence of 1 nM E₂. Antiestrogens were introduced at the time of transfection. Tamoxifen and ICI 182,780 did not alter FR-α promoter activity in the absence of ER (results not shown). (FIG. 1B) The FR-α promoter-luciferase construct (400 ng) was transfected into BG-1 cells (10⁶) in the absence or in the presence of E₂ (1 nM) and antiestrogens were introduced at the time of transfection. (FIG. 1C) HeLa cells (10⁶) were transfected with the thymidine kinase (TK)-promoter-luciferase (200 ng) or Rous Sarcoma Virus (RSV)-promoter-luciferase (200 ng) constructs and were co-transfected with either ER plasmid (25 ng) or with the plasmid vector (25 ng). Antiestrogens were introduced at the time of transfection.

FIGS. 2A and 2B are graphs showing an estrogen and antiestrogen dose response of a control ERE-driven promoter and the FR-α promoter in HeLa cells. The Y-axis in FIG. 2A indicates promoter activation, whereas the Y-axis in FIG. 2B indicates promoter repression. The cells (10⁶) were transfected with either ERE₂E1b-luciferase (100 ng) (FIG. 2A) or FR-α promoter-luciferase (100 ng) (FIG. 2B) and co-transfected with the ER expression plasmid (50 ng) where indicated. Cells that were not co-transfected with the ER-plasmid were co-transfected instead with the plasmid vector alone (50 ng). E₂, tamoxifen or ICI 182,780 were included from the time of transfection.

FIGS. 3A and 3B are graphs showing a time course of response of the control ERE₂E1b-luciferase (FIG. 3A) and FR-α promoter luciferase (FIG. 3B) to antiestrogens in HeLa cells. The cells (10⁶) were transfected with the indicated promoter-luciferase construct (100 ng) and co-transfected with ER expression plasmid (50 ng) in the presence of E₂ (1 nM). Twenty-four hours post transfection, tamoxifen or ICI 182,780 was introduced and at different intervals, cells were harvested to determine luciferase activity.

FIG. 4 is a graph showing a short-term reversibility of antiestrogen effects on the FR-α promoter. The antiestrogens, tamoxifen or ICI 182,780 were introduced 24 h after co-transfection of HeLa cells (10⁶) with the FR-α promoter-luciferase construct (100 ng) and the ER expression plasmid (50 ng). In one set of experiments, the antiestrogens were removed after a 6 h treatment. Cells were harvested 48 h post-transfection and assayed for luciferase activity.

FIG. 5 shows the effect of ER ligands on expression of the endogenous FR-α gene in HeLa-I-1 cells. The cells were either untreated or treated with E₂, ICI 182,780 or tamoxifen at the beginning of day 1 or day 3 and all of the cells were harvested at the end of day 6. The cell lysates were subjected to western blot analysis as described under Methods. The FR-α band intensities are indicated in relative units, using a value of 1 for the untreated control. The ER ligand treatment did not appreciably alter the viable cell count at the end of the 6-day period. FR-α expression was not altered by E₂ or antiestrogen treatment in the ER-negative parental HeLa cells (results not shown).

FIG. 6A is a functional mapping of the ER response element in the FR-α P4 promoter [Seq. ID No. 1] (FIG. 6A), the nucleotide sequence of the ER responsive P4 promoter fragment. An AP-1-like element [Seq. ID No. 2x] is highlighted and three G/C-rich (Sp1) elements [Seq. ID No. 3] [Seq. ID No. 4] [Seq. ID No. 5] are shown in bold print. The nucleotides are numbered in relation to the transcription initiation site (+1 nt). The translation start site is underlined.

FIG. 6B is a graph showing HeLa cells (10⁶) which were transfected with an FR-α promoter-luciferase construct (400 ng) in which the AP-1-like site (−154 to −143) [Seq. ID No. 2] was deleted. The cells were co-transfected with either ER expression plasmid (25 ng) or with the plasmid vector alone (25 ng). The transfected cells were grown in media containing E₂ (1 nM) and either in the absence of antiestrogen or in the presence of ICI 182, 780 or tamoxifen. The cells were harvested 48 h post-transfection for luciferase assays.

FIG. 6C is a graph showing HeLa cells (10⁶) which were transfected with FR-α promoter-luciferase (FR-α-Luc) (400 ng) [Seq. ID No. 6], FR-1-Luc in which the G/C-rich region (−47 nt to −18 nt) [Seq. ID No. 7] of the P4 promoter was replaced by the G/C-rich region in the SV40 early promoter (FR-α/SV40 (GC)₆) (400 ng), or with FR-α-Luc[Seq. ID No. 6] in which the initiator region (−28 nt to +33 nt) [Seq. ID No. 8] was replaced by the TATA box-containing initiator region of the SV40 early promoter (FR-α/SV40-lnr) (400 ng). ER expression plasmid (25 ng) or the plasmid vector (25 ng) was co-transfected as indicated. The transfected cells were grown in the presence of E₂ (1 nM) and either in the absence or in the presence of ICI 182, 780 for 48 h before harvesting them for luciferase assays.

FIGS. 7A, 7B, 7C and 7D show an electrophoretic mobility shift analysis (EMSA) of the interaction of ER and nuclear proteins in the G/C-rich region of the FR-α P4 promoter. A HeLa cell nuclear extract (HeLa NE) (10 μg) was used in all cases. ³²P-labeled probes corresponding to an FR-α promoter sequence (−89 nt to −50 nt) (FIGS. 7A and 7C), a 21-mer consensus Sp1 probe (FIG. 7B) or a 20-mer consensus ERE probe (FIG. 7D) were used (described under Methods). In Panel A, Lanes 3 and 4, 0.25 μg and 0.75 μg of ER were used, respectively. In all other reactions containing ER, 0.75 μg of the protein was used. The various EMSA conditions are indicated in the Figure and the assay procedure is described under Methods.

FIG. 8 is graph showing an effect of ERα vs. ERα on FR-α promoter activity. HeLa cells (10⁶) were transfected with the FR-α promoter-luciferase construct (200 ng) and co-transfected individually or in combination with expression plasmids (25 ng) for ERα, ERα or with the plasmid vector. The cells were treated from the time of transfection with E₂ (1 nm) and either without or with ICI 182, 780.

FIG. 9 shows an ER-dependent upregulation of FR-α by tamoxifen in HeLa cell tumor xenografts in vivo. Two weeks after sc injection of parental (FR+/ER−) or recombinant (FR+/ER+) HeLa cells, the tumor-bearing mice were given daily sc injections of tamoxifen (1 mg/kg) for 5 days and then sacrificed. Tumor cell lysates were subjected to Western blot analysis using anti-FR-α antibody. The figure shows a single representative data set.

FIGS. 10A, 10B, 10C and 10D are graphs showing the enhancement of FR-α promoter activity by testosterone/AR. In HeLa cells, AR was introduced by transiently transfecting an AR expression plasmid. FR-α promoter-luciferase, PSA-promoter-luciferase, or promoterless PGL3 basic-luciferase was co-transfected. Testosterone was introduced at the time of transfection (10 nM in FIGS. 10A, 10C, and 10D) and cells harvested for luciferase assays 48 h post-transfection. FIG. 10A shows promoter selectivity of testosterone/AR action. FIG. 10B shows testosterone dose response for the PSA and FR-α promoters. FIGS. 10C and 10D show AR dose responses for the FR-α and PSA promoters, respectively.

FIG. 11 is a graph showing the time course of testosterone/AR action on FR-α and PSA promoters. HeLa cells were cotransfected with AR expression plasmid and either FR-α promoter-luciferase or PSA promoter-luciferase. Testosterone (10 nM) was introduced 48 h post-transfection and the cells harvested to assay luciferase activity at the indicated times.

FIGS. 12A and 12B are graphs showing an upregulation of endogenous FR-α in HeLa cells by testosterone/AR (FIG. 12A) and potentiation of this effect by TSA (FIG. 12B). Where indicated, cells were transfected with AR expression plasmid and the testosterone (10 nM) and TSA (50 mg/ml) treatments were for 3 days post-transfection. FR-α expression was quantified by flow cytometry using a fluorescein-folic acid conjugate.

FIGS. 13A, 13B, 13C, 13D, 13E and 13F are graphs showing an enhancement of FR-α promoter activity by R5020/PR and comparison with the GRE₂e1b promoter. HeLa cells were co-transfected with each promoter-luciferase and expression plasmid for PRa or PRb and harvested at 72 h post-transfection for luciferase assays. The concentration of R5020 in panels C and D was 50 nM. FIGS. 13A and 13B: R5020 dose response for the action of PRa or PRb on FR-α promoter and GRE₂e1b promoter, respectively. FIGS. 13C and 13D: PR (a or b) dose response for R5020 action on the FR-α promoter and the GRE₂e1b promoter, respectively. FIG. 13E: Effect of RU486 on FR-α or GRE₂e1b promoter and on promoter activation by R5020 in HeLa cells transfected with PRa or PRb. FIG. 13F: Inability of R5020/PRa or R5020/PRb to activate the CMV promoter-luciferase co-transfected in HeLa cells.

FIGS. 14A and 14B are graphs showing the combined effect of PRa and PRb on the action of R5020 on the FR-α promoter (FIG. 14A) or the GRE₂e1b promoter (FIG. 14B). HeLa cells were cotransfected with either FR-α promoter-luciferase (FIG. 14A) or GRE₂e1b promoter-luciferase and the indicated amounts of PR expression plasmids for 72 h prior to harvesting for luciferase assays. The concentration of R5020 was 50 nM.

FIGS. 15A, 15B and 15C are graphs showing a time course and reversibility of R5020/PR action. HeLa cells were co-transfected with PR (a or b) expression plasmid and either FR-α promoter-luciferase or GRE₂e1b promoter-luciferase. FIGS. 15A and 15B: Time course of R5020 action on FR-α promoter and GRE₂e1b promoter, respectively. The time courses were initiated with the addition of R5020 (50 nM) 48 h post-transfection. FIG. 15C: HeLa cells were co-transfected with FR-α promoter luciferase and either PRa or PRb expression plasmid. In each case, R5020 was introduced at the time of transfection and withdrawn 6 h post-transfection. The cells were harvested for luciferase assay either at the time of R5020 withdrawal or 66 h later (i.e., at 72 h).

FIG. 16 is graph showing a potentiation of R5020 action on the FR-α promoter through the endogenous PR in T47D cells by SRC1 or TSA. T47D cells were transfected with FR-α promoter-luciferase and where indicated, co-transfected with SRC1 expression plasmid. R5020 (10 nM) and TSA (25 mg/ml) treatments were initiated at the time of transfection and lasted 3 days at the end of which the cells were harvested for luciferase assays.

FIGS. 17A and 27B are graphs showing an enhancement of FR-α promoter activity by dexamethasone. HeLa cells, which express endogenous GR, were transfected with FR-α promoter-luciferase or GRE₂e1b promoter luciferase. Cells were harvested for luciferase assays 96 h post-transfection. FIG. 17A: Dex dose response for enhancement of FR-α promoter activity. FIG. 17B: Time course of Dex action. Promoter activity was measured in terms of luciferase activity at the different time points. Dex treatment was either continued for 96 h or alternatively Dex was withdrawn at 48 h or at 72 h and the cells were harvested for luciferase assays at 96 h.

FIGS. 18A and 1 BB are graphs showing a combined effect of Dex and R5020 on FR-α promoter activity (FIG. 18A) and expression of the endogenous FR-α (FIG. 18B) in HeLa cells. FIG. 18A: HeLa cells (untransfected or transfected with FR-α promoter luciferase) were treated with R5020 alone, Dex alone, or both R5020 and Dex for 96 h before harvesting for luciferase assays. FIG. 18B: Recombinant HeLa cells, stably expressing PRb were treated with Dex, R5020, or both Dex and R5020 for 4 days. The change in endogenous FR-α expression was measured by flow cytometry using a fluorescein conjugated folic acid probe.

FIGS. 19A and 19B are graphs showing a mapping the FR-α promoter regions responsive to R5020/PRa, R5020/PRb, Dex/GR and testosterone/AR. FIG. 19A: the FR-α promoter fragments (indicated) attached to luciferase, were transfected into HeLa cells. For the R5020 experiments, the cells were co-transfected with PRa or PRb expression plasmids and the increase in luciferase activity measured after 72 h of treatment with R5020. Dex treatment of HeLa cells (which have endogenous GR) was for 96 h post-transfection (no co-transfection). FIG. 19: Effect of testosterone on the promoter activity of various FR-α promoter-luciferase 5′ deletion constructs transfected into HeLa cells co-transfected with AR expression plasmid. The cells were harvested 48 h post-transfection for the luciferase assays.

FIGS. 20A, 20B and 20C show upregulation of FR-α in HeLa cells in vitro and in mouse tumor xenografts of HeLa cells in vivo by treatment with Dex. FIG. 20: HeLa cells in culture were treated with either vehicle (alcohol) alone or with the indicated concentrations of Dex for 96 h and then harvested and the cell lysates were subjected to western blot analysis using anti-FR antibody to detect FR-α. FIG. 20B: HeLa cells were treated in vitro with vehicle (alcohol) or for the indicated periods with Dex (1 μM). The cell lysates were analyzed for FR-α by western blot as in FIG. 20A. FIG. 20C: Two groups of three female SCID mice were implanted subcutaneously with HeLa cells to generate tumor xenografts. In one group of mice, slow release Dex pellets (one 0.001 mg Dex pellet per mouse) were implanted subcutaneously, seven days prior to sacrificing the mice to harvest the tumors. In the placebo group, the mice were implanted subcutaneously with placebo pellets. At the end of the treatment, the tumor cell lysates were analyzed for FR-α by western blot.

### DETAILED DESCRIPTION OF THE PREFERRED EMBODIMENTS

### Definitions

The terms “treating,” “treatment,” and “therapy” as used herein refer to curative therapy, prophylactic therapy, and preventative therapy. The term “diagnostic” and “diagnosis” as used hereinafter refer to screening individuals for cancer, monitoring a cancer in patients and whole body and other imaging of tumors.

The present invention provides a method of treating patients with various solid tumors. The present method includes administering to an individual at least one FR-α inducer or combinations of FR-α inducing agents and a FR-specific diagnostic or therapeutic system. The present invention incorporates the following discoveries:

(a) FR-α is expressed on the surface of a wide variety of cancer cells in solid tumors.

(b) In contrast to tumor tissues, FR-α expressed in normal tissues is inaccessible to the blood stream.

(c) A significant fraction of FR-α expressed on tumor cells is shed into extracellular fluids such as blood, ascites and cerebrospinal fluid.

(d) The soluble form of FR-α shed into extracellular fluids can be detected and quantified by assays using fluorescent folate or other conjugates or FR-specific antibodies.

(e) Solid tumors expressing FR-α may be visualized using tumor imaging agents that bind specifically to FR-α.

(f) FR-α binds specifically and possibly internalizes folate conjugates, which include but are not restricted to folate conjugates of cytotoxics, radiopharmaceuticals and antibodies, folate-coated liposome drug carriers and folate-tethered nanoparticles as well as antifolate drugs and various immunotherapeutic agents.

(g) The expression of FR-α may be increased substantially and selectively in tumors using agents that act through various steroid hormone receptors as well as inhibitors of HDAC, making virtually all modalities of FR-mediated cancer diagnostics and therapeutics viable options in the clinic.

The discovery that FR-α is regulated by steroid hormone receptors and that this regulation is restricted to FR-positive tissues is useful to produce a uniform high level of expression of this protein concomitantly with the use of targeted diagnostic and therapeutic agents. Thus, in one aspect, the present invention relates to manipulating FR-α expression in cancer cells in order to render FR-mediated diagnostics and therapeutics particularly useful in the treatment of patients.

### The Folate Receptor

Structural and functional diversity and distribution in normal and malignant tissues. The folate receptor (FR) is a glycopolypeptide with a high affinity for folic acid and the circulating form of folate, (6S) N⁵-methyltetrahydrofolate (K_{D}<10⁻⁹M) (1). Most normal tissues virtually lack FR. A relatively high level of FR was originally reported in placenta, choroid plexus, proximal kidney tubules, and KB epidermoid carcinoma cells (2-7). Certain extracellular fluids (milk, cerebrospinal fluid, amniotic fluid) and KB cell culture media showed significant levels of a soluble high-affinity folate binding protein (sFBP) (7-13) apparently derived from a membrane-associated precursor (FR) (14-16). While placental FR is the likely source of sFBP in umbilical cord serum, normal serum contains virtually no sFBP.

From cDNA cloning, three folate receptor isoforms, type α (17, 18), type β (19) and type γ (20, 21) were identified. FR-α (18) and FR-β (22) are attached to the cell surface by a glycosyl-phosphatidylinositol (GPI) membrane anchor, whereas, FR-γ is constitutively secreted due to the lack of a signal for GPI anchor attachment (21). The FR-α gene is polymorphic due to a mutation resulting in a truncated polypeptide termed FR-γ′ (20, 23). The membrane anchored FR-α (24, 25) and FR-β(26) can mediate internalization of receptor bound folate compounds and folate conjugates. FR-α and FR-β also show differences in their relative affinities and stereospecificities for folate compounds and antifolates (27) attributed to specific differences in their primary structure (28, 29).

The FR isoforms exhibit narrow tissue and tumor specificities. Among normal tissues, FR-α is expressed in epithelial cells of the placenta, breast, lung, kidney proximal tubules, choroid plexus, ovary, fallopian tubes, uterus, endocervix and salivary glands (30-32, 33). GPI-anchored proteins such as FR-α are typically expressed on the apical (luminal) surface of polarized epithelial cells. Among malignant tissues, FR-α is consistently expressed in non-mucinous adenocarcinomas of the ovary, uterine and cervical adenocarcinomas, testicular choricarcinomas and certain brain tumors and less frequently in breast, colon and renal carcinomas (30-32, 33, 34, 35, 36, 37). FR-β is highly expressed in placenta (19), in mature neutrophils (38) (where it is unable to bind folate, 39), in activated monocytes and macrophages (40), and in more than half of all acute myeloid leukemias (38, 39). The secreted FR-γ is virtually undetectable in normal serum but may be elevated in certain lymphoid malignancies (21).

Quantitative analysis by flow cytometry (41) and quantitative in situ hybridization (33) have established that despite consistent patterns of FR-α expression in normal and malignant female reproductive tissues, the actual level of expression of the receptor varies among tumors from different individuals and is also heterogeneous within the same tumor; this variability occurs in a range up to two orders of magnitude. All of the successful FR-α-targeted therapies in animal models described in a later section have used cell lines (e.g., KB cells) that uniformly express FR-α at levels close to the high end of this range.

Physiologic significance of FR-α. FR-α is capable of transporting folate into the cell but other folate uptake pathways are generally used by adult tissues. Since FR-α expression in normal adult tissues is restricted to the apical surface of epithelial cells where it is not in contact with the circulation, the physiologic role of FR-α is clear only in specific instances. In placenta, FR-α is required for trans-placental folate uptake by the fetus (2). In the kidney, FR-α may be required for urinary clearance of folate (42). Gene knock-out studies have shown that FR-α is essential for nerve tube development in the absence of very high levels of folate (43). FR-β gene knockout is not lethal but results in increased risk of developmental defects due to impairment of folate transport in the embryo (44).

Clinical significance of FR. Among the available tumor targets/markers, FR has certain distinctive advantages in that (i) it binds to a ligand (folate) with a high affinity; (ii) FR quantitatively recycles between the cell surface and intracellular compartments effectively internalizing folate/antifolate compounds and folate conjugates and (iii) FR-α expression in proliferating normal tissues (other than placenta) is restricted to the luminal surface of certain epithelial cells where it is inaccessible to the circulation whereas it is consistently expressed in specific types of major malignant tumors where it is accessible via the circulation. The occurrence of multiple, functionally distinguishable, tissue-specific FR isoforms provides an added level of tissue selectivity in FR-mediated cancer therapy/prognosis/diagnosis.

Many innovative strategies for targeting FR continue to appear in the literature. Radionucleide conjugates of folic acid are effective in whole body imaging of ovarian cancer in humans and have been developed by Endocyte Pharmaceuticals Inc. for clinical use (45). In recent pre-clinical and clinical studies, FR-α has shown considerable promise as a potential means of delivering a wide variety of novel therapeutic agents, including genes, to tumor cells and as a tumor and serum marker (Reviewed in 45-49). The novel therapeutic approaches include the use of folate or antibody conjugates of cytotoxics (50-52), and radiopharmaceuticals (53-58), folate-coated liposomes containing antisense oligonucleotides, genes or cytotoxics (59-65), folate conjugates of pro-drugs (66) or a pro-drug activating enzyme (67) or folate-linked nanoparticle carriers for therapeutic drugs and genes (68-71). In cells expressing high levels of FR-α, the receptor also offers the preferred uptake route for novel antifolate drugs, which target glycineamide ribonucleotide formyltransferase and thymidylate synthase (72); antifolate drugs that are selectively transported by FR have recently been developed (73a, 73b). A variety of immunological therapies have also been developed. Bifunctional antibodies, which bind FR-α and T-cell antigens, have induced a profound immune response against tumors in xenogenic animal models (74, 75) and in patients with advanced ovarian cancer (76-78). Similarly, a chimeric molecule consisting of interleukin 2 and a single-chain Fv of an antibody against FR-α effectively inhibited tumor growth in vivo (79). FR may also be used to produce DNA and polypeptide vaccines against tumor cells (80-85). Very recently, strong in vivo antitumor responses were obtained using dual-specific T-cells (86) and by targeting immunogenic haptens to FR-α (87).

The high success rate of the FR-α-targeted approaches in animal models and the promise shown in early clinical trials in terminal patients is very encouraging. However, the animal studies have the advantage that the model tumors express uniform high levels of FR-α, in contrast to human cancer. All of the above FR-α-targeted approaches should therefore greatly benefit from new strategies to upregulate the receptor in the tumors.

### Upregulation of FR-α

Expression of FR-α in the cancer cells is accomplished by administering a pharmaceutical composition comprising a biologically effective amount of steroid receptor agonist or antagonist to the subject. A useful estrogen receptor antagonist is tamoxifen. Other antagonists are described in McDonnell, D. P. (1999) Trends Endocrinol. Metab. 10:301-311, which is specifically incorporated herein by reference. In another embodiment, biologically effective amounts of an estrogen receptor antagonist and a histone deacetylase inhibitor are administered to the subject. A useful histone deacetylase inhibitor is TSA. Other histone deacetylase inhibitors are described in Curr. Opin. Oncol. 2001 November: 13(6): 477-83. Review. PMID: 11673688 [PubMed—indexed for MEDLINE]; Yoshida M, Furumai R, Nishiyama M, Komatsu Y, Nishino N, Horinouchi S. Related Articles Histone deacetylase as a new target for cancer chemotherapy. Cancer Chemother. Pharmacol. 2001 August: 48 Suppl. 1:S20-6. Review. PMID: 11587361 [PubMed—indexed for MEDLINE] Jung M. Related Articles Inhibitors of histone deacetylase as new anticancer agents. Curr. Med. Chem. 2001 October: 8(12):1505-11. Review. PMID: 11562279 [PubMed—indexed for MEDLINE]; all of which are specifically incorporated herein by reference. In other embodiments, agonists of the progesterone receptor (e.g., progestin), the androgen receptor (e.g., testosterone, dihydroxytestosterone) or the glucocorticoid receptor (e.g., dexamathasone) are administered to increase FR-α expression in the tumor cells. In additional embodiments, the various steroid receptor antagonists and agonists are administered in different combinations with or without histone deacetylase inhibitor to effect an increase in FR-α in the tumor cells.

In addition, various vitro tools and methods are suitable for assaying the effects of the various steroid receptor agonists or antagonists and histone deacetylase inhibitors on upregulation of FR-α. These include established cell lines as well as stably transfected cells. In addition, primary cell cultures may be used, and maintained as short-term cultures with 20% FBS as the only folate source, and in the presence of growth factors.

Rabbit antisera and a monoclonal antibody against FR-α are used in flow cytometry and Western blot assays for measuring receptor expression levels. A number of assays quantifying FR-α expression levels in cells are standard and include Western blot for detecting the presence of FR-α protein, Northern blot for detecting the presence of FR-α transcript, immunocytochemical staining for microscopic visualization of FR-α cellular distribution, multi-color flow, quantitative real time RT-PCR analysis of the FR-α transcript, quantitative in situ hybridization analyses and radioactive (using [³H] folic acid) and fluorescent ligand (using a FITC-folate conjugate) binding assays.

### Administration of the Pharmaceutical Composition

The pharmaceutical compositions that contain the steroid receptor agonist or antagonist and histone deacetylase inhibitor may be administered concurrently, separately, in combination, or in a sequential manner, depending on the most efficacious treatment modality for the individual agents. The route of administration may be oral or by injection, depending on the most efficacious treatment modality for the individual agents. The number of treatments, the period of administration and the dose of the individual agents will also be determined based on the most efficacious treatment modality for the individual agents.

The pharmaceutical compositions are administered once or repeatedly in an effective amount. As used herein, the term “effective amount” means the total amount of each active component of the pharmaceutical formulation or method that is sufficient to show a meaningful subject or patient benefit, i.e., a significant increase in the amount of FR-α on the surface of tumor cells (for targeted cancer therapeutics and tumor imaging) or in extracellular fluids, including blood (for diagnostics).

Those skilled in the art will recognize that delivery via local injection contemplates the use of a syringe, catheter or similar device, which delivers the pharmaceutical composition to the target site, i.e., to an area exhibiting cellular proliferative disease. Delivery may be direct, i.e., intratumoral, or nearly direct, i.e., intralesional, that is, to an area that is sufficiently close to a tumor so that the active agent exhibits the desired pharmacological activity with respect to the tumor itself. Thus, in one aspect, the pharmaceutical composition is preferably delivered intralesionally or intratumorally. In another aspect, the pharmaceutical composition is delivered intravenously i.e., systemically.

When a therapeutically effective amount of the pharmaceutical composition used in the method of the invention is administered by injection, the pharmaceutical composition will preferably be in the form of a pyrogen-free, parenterally-acceptable, aqueous solution. The preparation of such parenterally-acceptable solutions, having due regard to pH, isotonicity, stability, and the like, is within the level of ordinary skill in the art of pharmacology. A preferred pharmaceutical composition for injection should contain, in addition to the vector, an isotonic vehicle such as Sodium Chloride Injection, Ringer's Injection, Dextrose Injection, Dextrose and Sodium Chloride Injection, Lactated Ringer's Injection, phosphate buffered saline (PBS), or other vehicle as known in the art. The pharmaceutical composition used in the method of the present invention may also contain stabilizers, preservatives, buffers, antioxidants, or other additives known to those of skill in the art.

The duration of treatment with the pharmaceutical composition used in the method of the present invention will vary, depending on the unique characteristics of the pharmaceutical composition and the particular effect to be achieved, the severity of the disease being treated and the condition and potential idiosyncratic response of each individual patient. Ultimately the attending physician will decide on the appropriate duration of therapy with the pharmaceutical composition used in the method of the present invention.

The invention may be better understood by reference to the following examples, which serve to illustrate but not to limit the present invention.

### Examples

A skilled artisan can readily recognize that there is no limit as to the structural nature of the agents of the present invention. As such, without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

DNA Constructs: Construct design used either natural restriction sites or restriction sites created by the polymerase chain reaction (PCR) using Vent DNA polymerase (New England Biolabs) and custom oligonucleotides from Gibco BRL. In cases where a natural restriction site could not be found for use, complementary primers containing an appropriate restriction site or point mutation were used in conjunction with upstream and downstream primers containing restriction sites. Alternatively, mutagenic oligonucleotides were used as end primers to amplify the desired fragment. The PCR products were first digested at both ends with the appropriate restriction enzymes and cloned into the PGL3-basic plasmid (Promega) or subcloned into the FR-\tilde{α}promoter construct (−3394 nt to +33 nt, corresponding to the transcription initiation site at +1 nt) [Seq. ID No. 6] inserted into the PGL3 basic plasmid at Mlu I and Xho I sites of the polylinker. The FR-α/SV40(GC)₆-Luc construct was generated using the upstream primer 5′-GTCAGCATATGTAGTCCCGCCC-3′ [Seq. ID No. 9] containing a synthetic restriction site (Nde I) and the downstream primer 5′-AAACTTAAGCAGCGATGGGGC-3′ [Seq. ID No. 10] containing a synthetic restriction site (Afl III) corresponding to regions in the SV40 promoter of the pGL3-control plasmid (Promega). The FR-α/SV40-lnr construct was generated using the upstream primer 5′-ATTCTCCGCGGCATCGCTGAC-3′ [Seq. ID No. 11] containing a synthetic restriction site (Sac II) corresponding to a region in the SV40 promoter and the downstream primer 5′-CACTGCATACGACGATTCTGTG-3′ [Seq. ID No. 12] corresponding to a region in the luciferase gene of the pGL3-control plasmid. The downstream restriction site (Nar I) used in the subcloning occurred naturally in the plasmid. The recombinant plasmids were amplified in XL1Blue and purified using the Qiagen plasmid kit (Qiagen), or by CsCl gradient centrifugation followed by phenol chloroform extraction and ethanol precipitation. The entire cloned sequence was verified using the Beckman CEQ 2000 automated sequencer. The PCR reaction for sequencing was carried using the DTCS kit from Beckman.

Cell Culture and Transfection: HeLa I-1 cells were kindly provided by Dr. S. T. Rosen. BG-1 cells were provided by Dr. Randolf Ruch. HeLa (American Type Tissue Collection) and HeLa I-1 cells were routinely cultured in phenol red-free MEM supplemented with fetal bovine serum (FBS) (10%), penicillin (100 units/mL), streptomycin (100 mg/mL), and L-glutamine (2 mM). BG-1 and IGROV1 (American Type Tissue Collection) cells were routinely cultured in DMEM and supplemented as above. Treated or transfected cells were grown in phenol-red free media supplemented with charcoal-stripped FBS (5% v/v), penicillin (100 units/mL), streptomycin (100 mg/mL), L-glutamine (2 mM), insulin (2 μg/mL), and transferrin (40 μg/mL) unless otherwise noted. In addition, treated HeLa I-1 cells were grown in culture with 50 μg/mL G418. 17α-estradiol (E₂), Tamoxifen (TAM) or ICI 182,780 (ICI) were used where indicated at the concentrations specified. Transfections with the cDNA constructs were carried out in six-well plates (Corning) using lipofectamine (Gibco, BRL) or FuGene (Roche Diagnostics), according to the manufacturer's suggested protocol. Uniformity of transfection was routinely monitored through cotransfections with the β-galactosidase expression plasmid, pSV-β-gal (Promega). β-galactosidase activity was measured colorimetrically using the assay system available from Promega.

Luciferase Assay: Forty-eight hours after transfection, the cells were washed once with PBS (10 mM sodium phosphate, pH 7.5, 150 mM NaCl) and harvested in 400 μL of reporter lysis buffer provided with the luciferase assay system (Promega). The samples were centrifuged at 12,000 g for 2 min at room temperature. The supernatant was assayed for luciferase activity in a luminometer (Lumat LB9501, Berthold) using the luciferase substrate from Promega.

Electrophoretic Gel-mobility Shift Assay (EMSA): EMSA grade HeLa cell nuclear extract was purchased from Promega. Equimolar quantities of complementary oligonucleotides corresponding to specific regions in the FR-α P4 promoter, a consensus Sp1 sequence probe (5′-ATTCGATCGGGGCGGGGCGAG-3′, Promega) [Seq. ID No. 13] or an ERE sequence probe (5′-GTCAGGTCACAGTGACCTGA-3′, Invitrogen) [Seq. ID No. 14] were denatured in TE buffer (100 mM Tris-Cl, 10 mM EDTA, pH 7.5) at 100° C. for 5 min and annealed by cooling to room temperature in a thermal cycler at a rate of −1° per minute. The oligonucleotides were labeled using [α³²P] ATP and T4 polynucleotide kinase (Promega). 10 μg of nuclear extract was incubated with ³²P labeled probes (40,000 cpm) in 10 μL of binding solution [25 mM HEPES buffer, pH 8.0, containing 50 mM KCl, 0.5 mM MgCl₂, 0.5 mM DTT, 2 mg of poly(dl-dC)-poly(dl-dC) and 10% glycerol]. Recombinant ER was added to particular samples in the amounts indicated. The samples were incubated at room temperature for 15 min. After addition of the appropriate antibody in certain reaction tubes, the samples were incubated at room temperature for an additional 30 min. The reaction mixture was then run on a 4% polyacrylamide gel at 275 volts for 45 minutes. The resulting gel was subjected to either autoradiography on X-ray film or phosphorimaging on a phosphor screen (Molecular Dynamics) using a Storm 840 scanner (Molecular Dynamics) and ImageQuant version 1.1 imaging software (Molecular Dynamics).

Western Blots: The samples were electrophoresed on a sodium dodecyl sulfate-polyacrylamide gel and transferred electrophoretically to nitrocellulose. The blots were probed with a rabbit antibody to FR-α and the receptor visualized using a chemiluminescence kit (Amersham) following the protocol provided by the vendor. The densitometric scans of the blots were analyzed using the ImageQuant software (Molecular Dynamics).

### Results

FR-α promoter usage in model cell lines: The full promoter region of the FR-α gene contains two basal promoters (P1 and P4) with distinct transcription initiation sites but the predominant mRNA species detected in malignant cells by RNase protection assay is the P4 promoter-driven transcript. Since the inventor herein used transient transfections to study the FR-α promoter activity, it was of importance to first test whether this promoter preference of the endogenous FR-α gene is reflected in the promoter-luciferase reporter constructs during transient transfection of the model cell lines used (i.e., HeLa cervical carcinoma, IGROV-1 ovarian carcinoma and BG-1 ovarian carcinoma). As shown in Table 1, the FR-α-P1-promoter-luciferase activity was much lower than that of P4-luciferase, and the FR-α promoter activity was greatly diminished by deleting the Sp1 elements in the P4 promoter, indicating that the promoter preference of the chromosomal gene is retained in the model cell lines even outside the chromosomal context.

TABLE 1
FR-α promoter usage in model cell lines
Activity of Promoter Luciferase Reporter Constructs^{a}

| Cell line | P4^{b}/P1^{c} | Full promoterΔ P4^{d}/Full promoter^{e} |
|---|---|---|
| HeLa | 15.06 | 0.2120 |
| IGROV-1 | 11.26 | 0.1343 |
| BG-1 | 4.48 | 0.1350 |

^{a}The transcription initiation site in the P4 promoter is designated +1 nt. The results represent at least three independent experiments in which 500 ng of the following constructs were transfected into HeLa, IGROV-1 or BG-1 cells (10⁶). Percent error is less than 5.
^{b}FR-α P4 promoter fragment (−271 nt to +33 nt) [Seq. ID No. 1]
^{c}FR-α P1 promoter fragment (−3394 nt to −2468 nt) [Seq. ID No. 15]
^{d}FR-α gene fragment spanning both P1 and P4 promoters (−3394 nt to +33 nt) with a deletion of the Sp1 sites in the P4 promoter [Seq. ID No. 16]
^{e}FR-α gene fragment spanning both P1 and P4 promoters (−3394 nt to +33 nt) [Seq. ID No. 6]

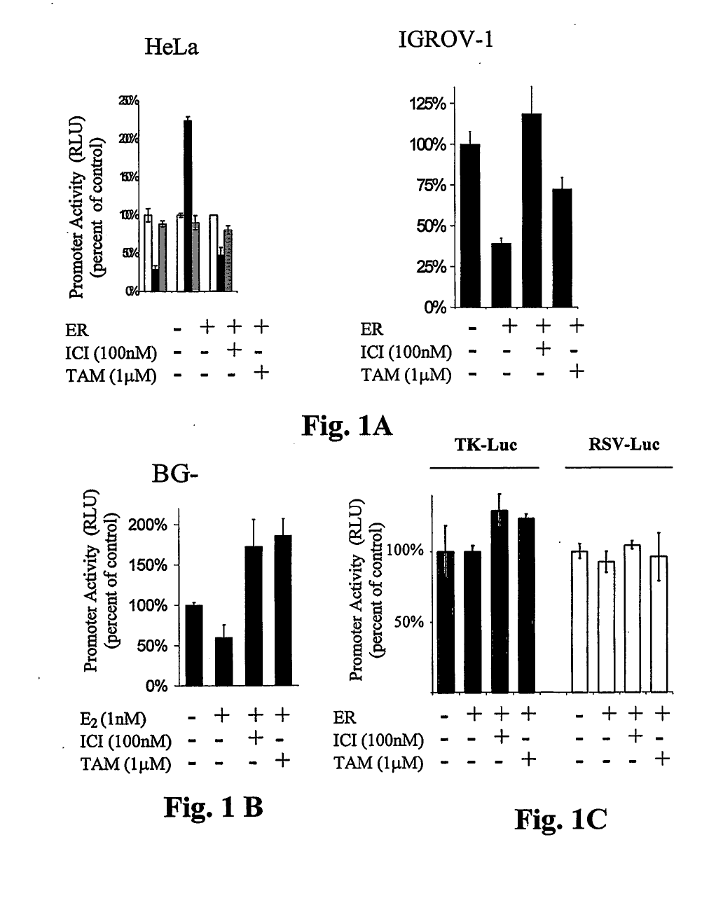

ER-mediated repression of the FR-α promoter and derepression by antiestrogens: Co-transfection of HeLa and IGROV-1 cells with a plasmid expressing ERα and the FR-α promoter-luciferase reporter plasmid caused 71 percent and 61 percent reduction in the promoter activity, respectively (FIG. 1A); the promoter activities were either partially or completely restored to the uninhibited values in the presence of pharmacologic concentrations of the antiestrogens, tamoxifen or ICI 182,780 (FIG. 1A). To ensure that the endogenous ER expressed in malignant cells can modulate the FR-α promoter, BG-1 ovarian carcinoma cells, which express endogenous ER, were transfected with FR-α promoter-luciferase. As expected, treatment with ICI 182,780 increased the promoter activity in BG-1 cells (FIG. 1B). It may be noted that in FIG. 1B, the promoter activity in the presence of antiestrogens exceeded the value in the absence of E₂. As seen below, this may be ascribed to the repressive effect of ER even in the absence of E₂.

The ER and ER ligand response of the FR-α promoter was clearly promoter-specific since other promoters (RSV and TK promoters) did not respond appreciably to ER either in the presence or in the absence of antiestrogens under the cell culture and transfection conditions used in this study (FIG. 1C).

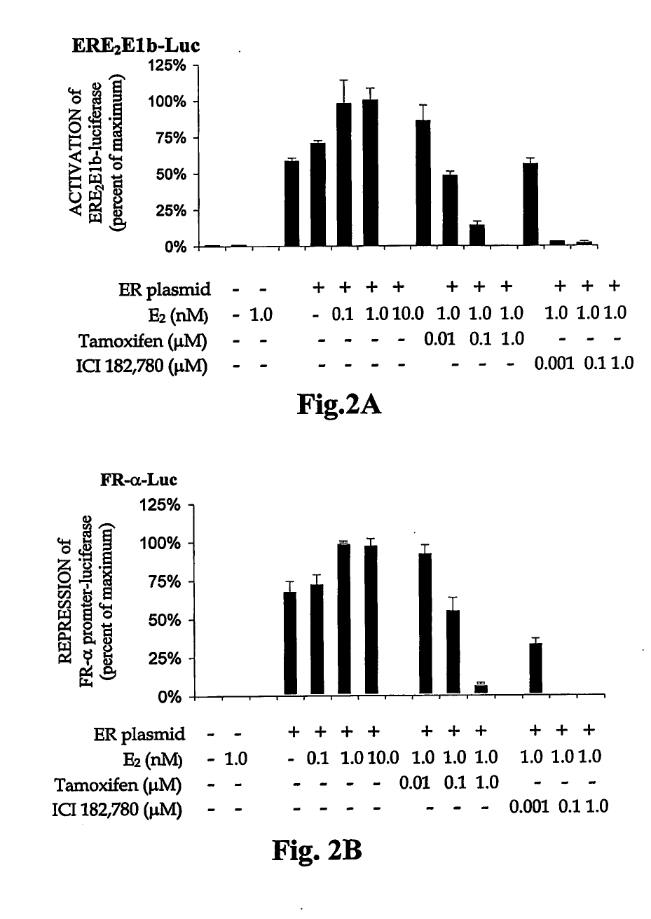

ER ligand dose response of the FR-α promoter: E₂ activates ERE-driven promoters at a sub-nanomolar concentration; the estrogen antagonist, tamoxifen, which has approximately 100-fold lower affinity for ER compared with E₂, generally counteracts the estrogen effect at a higher relative concentration whereas the pure antiestrogen, ICI 182,780 effectively counteracts the E₂ effect at the lower concentration. Such a ligand dose response is a hallmark of ER-mediated transcriptional effects. Contrary to the ERE-mediated effect on a control ERE-driven promoter (ERE₂E1b) (FIG. 2A) the FR-α promoter was repressed by E₂ and the repression was counteracted by tamoxifen and ICI 182,780 (FIG. 2B). (In FIG. 2. the Y-axis in FIG. 2A indicates promoter activation, whereas the Y-axis in FIG. 2B indicates promoter repression). The dose responses of the ligand effects on the FR-α promoter, however, occurred within concentration ranges comparable to those required to regulate the ERE₂E1b promoter. It may also be noted that the ER effects on both the ERE₂E1b promoter and the FR-α promoter occurred (albeit suboptimally) even in media containing charcoal-stripped FBS, suggesting that the unliganded receptor may also repress the FR-α promoter and that this effect is enhanced by E₂.

ER dose response for repression of the FR-α promoter: The level of ER in various tumors is variable but, in general, all ER+ tumors have adequate ER to mediate the classical physiologic effects of estrogen. In view of the benefit of modulating the FR-α gene with antiestrogens, it was important to compare the ER dose dependence for modulating the FR-α promoter with that of a classical ERE-dependent gene. Table 2 shows the degree of activation of an ERE-dependent promoter luciferase or repression of FR-α promoter-luciferase in response to co-transfection with different amounts of the expression plasmid for ER in the presence of E₂. The similarity in the ER plasmid dose response of the two promoters supports the view that ER repression of the FR-α promoter occurs at ER levels comparable to those required for activation of a classical estrogen-responsive gene.

TABLE 2
ER dose response of the FR-α promoter

| ER plasmid^{a} | Repression of | Activation of |
|---|---|---|
| (ng/10⁶ cells) | FR-α promoter^{b} | ERE₂E1b-promoter^{c} |
| 0 | 0 | 0 |
| 2 | 11 ± 0.9 | 6 ± 0.4 |
| 10 | 66 ± 10.5 | 85 ± 4.3 |
| 50 | 100 | 100 |

^{a}ER plasmid in the amounts indicated were cotransfected with either the FR-α promoter-luciferase construct (500 ng) or the ERE₂E1b-promoter-luciferase construct (500 ng).
^{b}Percent of maximum repression
^{c}Percent of maximum activation

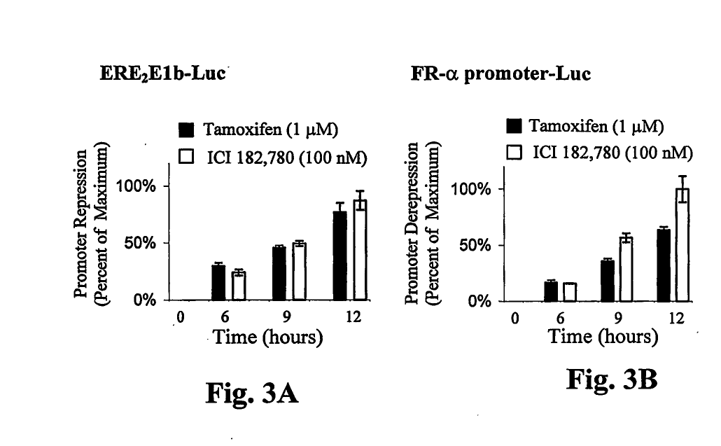

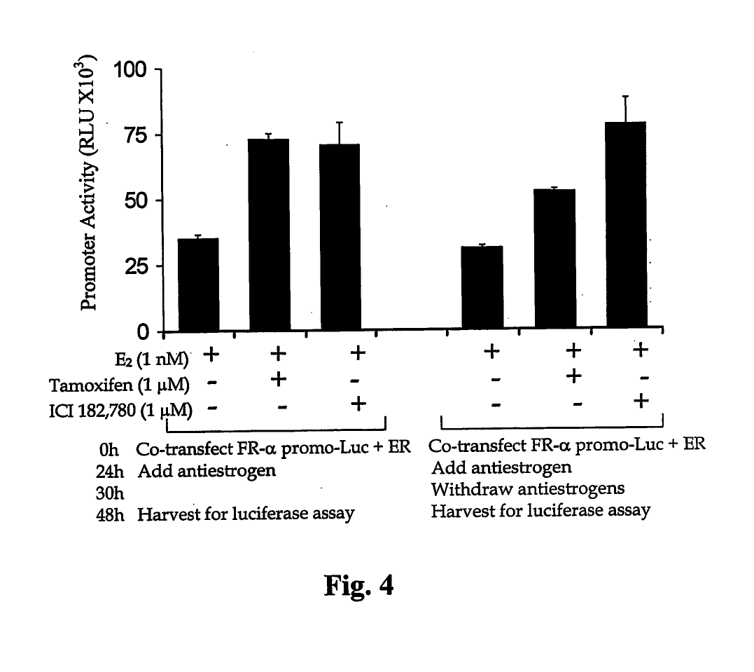

Time course and reversibility of antiestrogen effects on the FR-α promoter: When antiestrogens (tamoxifen and ICI 182,780) were introduced 24 h after transfection with ER and either the ERE₂E1b-luciferase (FIG. 3A) or the FR-α promoter-luciferase (FIG. 3B), the repression or activation respectively of the promoters began within hours and progressively increased. As seen in FIG. 4, even when antiestrogens were introduced 24 h after co-transfection of cells with FR-α promoter-luciferase and ER, there was a doubling of the reporter activity at the end of 48 h post-transfection. A brief 6 h treatment with ICI 182,780 was adequate to produce full derepression of the FR-α promoter which lasted for at least 18 h after withdrawing the compound (FIG. 4). In contrast, the derepression of the FR-α promoter by tamoxifen was partially lost after withdrawing the compound. This result is entirely consistent with the known modes of ER-mediated actions of tamoxifen and ICI 182,780 in vivo; tamoxifen acts by reversibly modulating ER whereas the net result of the interaction of ICI 182,780 with ER is by down-regulation of the receptor.

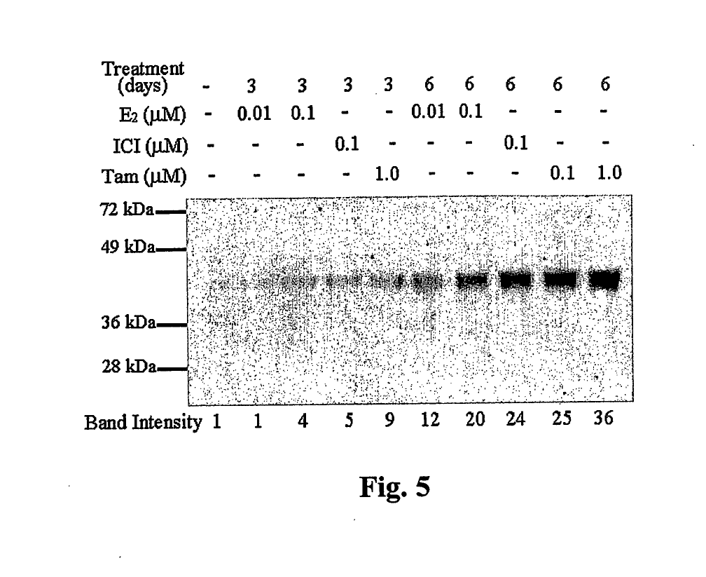

Modulation of endogenous FR-α expression by ER: Transcriptional effects observed in model promoter-reporter constructs commonly translate into more pronounced effects in the corresponding endogenous genes, in the chromosomal context. The relatively low transcription rate of the FR-1 gene together with the relatively slow mRNA and protein turnover rates (˜24 h) of FR-α (1a) confound efforts to directly quantitate the change in endogenous FR-α expression caused by transfected ER during the short-term (2 or 3 days) transient transfections. Furthermore, only a small fraction of the FR-α⁺ cells (<10 percent) may be transfected in transient transfections. Given the above considerations, even profound transcriptional effects on the endogenous FR-α gene may be expected to result in very small changes in FR-α levels against a high background in transient transfection systems. To examine the effect of ER/ER ligands on endogenous FR-α expression, it is therefore imperative to use a cell culture system in which all of the cells express ER over an extended period. Since restoration of wild-type ER expression would adversely affect the viability of established (FR-α⁺) cell lines, the inventor used a stable recombinant HeLa cell line (HeLa I-1) (2a) expressing a mutant (G400V) form of ER that has a lower affinity for E₂. The mutant ER allows long-term cell survival in culture but may have altered E₂ response characteristics due to different E₂-induced conformations of the protein compared to wild-type ER. As seen from the western blot in FIG. 5, treatment of HeLa-I-1 cells with either tamoxifen or ICI 182,780 resulted in a high level of endogenous FR-α (up to ˜36 fold over the untreated control) over a period of six days. Consistent with the anticipated alteration in E₂ response of the mutant ER in Hela-I-1 cells, E₂ also produced an increase in FR-α, . . . notably at supra-physiologic concentrations, expected from the decreased affinity of the mutant ER for E₂ (FIG. 5). The antiestrogen effects occurred at pharmacologic concentrations of the ligands. These results underscore a profound effect of ER and ER ligands on the endogenous FR-α gene expression.

To ensure that ER ligands will modulate FR-α expression selectively in FR-α positive and ER-positive tissues and that the ligands will not alter the tissue specificity of FR-α expression, tamoxifen and ICI 182,780 were tested on a variety of ER⁺/FRα-negative cells including the ER⁺ BG-1 cells discussed above. In no case did the inventor observe induction of endogenous FR-α expression (results not shown). It appears that ER will only modulate the endogenous FR-α gene in cells in which the gene is transcriptionally active.

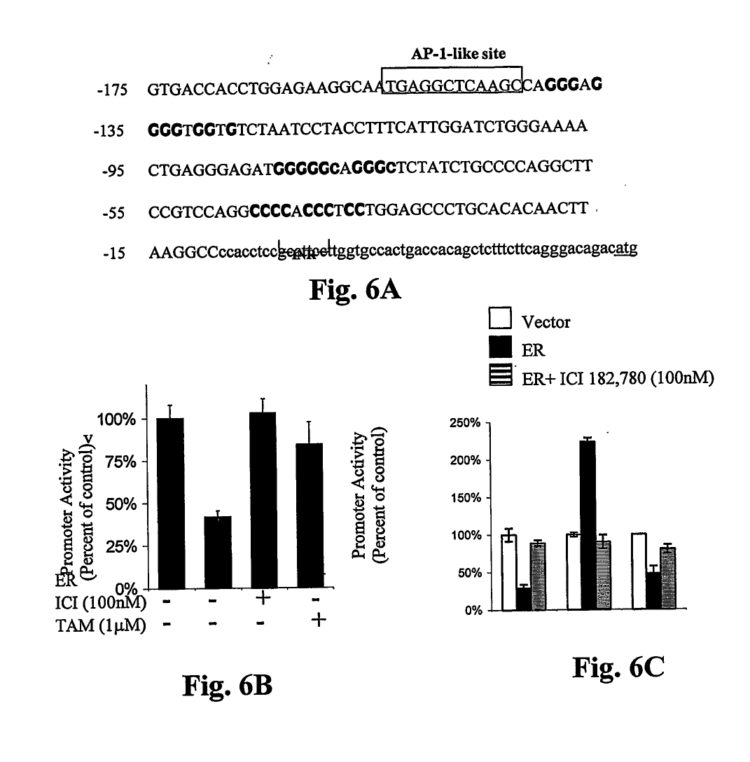

Mapping the ER-responsive element in the FR-α promoter: A short FR-α fragment (−173 nt to +33 nt) (FIG. 6A) [Seq. ID No. 1] containing the basal P4 promoter retained the ER ligand response of the FR-α promoter in HeLa and IGROV-1 cells (results not shown). This fragment lacks a classical estrogen response element (ERE). The fragment, however, does contain two cis-elements known, in other genes, to mediate an ER ligand response, i.e., an AP-1-like element [Seq. ID No. 2] and a cluster of three G/C-rich (Sp1 binding) elements (FIG. 6A) [Seq. ID No. 3] [Seq. ID No. 4] [Seq. ID No. 5]. Deletion of the AP-1-like element failed to abrogate the ER modulation of the FR-α promoter (FIG. 6B) excluding a role for AP-1 in mediating the ER effect. Since deletion of the G/C-rich region [Seq. ID No. 7] would abolish the basal promoter activity of the TATA-less P4 promoter, the entire cluster of Sp1 elements was substituted with a G/C-rich region from the heterologous SV40 early promoter. The resulting chimeric construct retained promoter activity but was activated by E₂ in the presence of ER; this activation was counteracted by ICI 182,780 in striking contrast to the ER modulation of the native P4 promoter [Seq. ID No. 1] (FIG. 6C). These results pinpoint the G/C-rich region [Seq. ID No. 7] in the FR-α P4 promoter as an essential site for the action of ER ligands. FIG. 6C also shows that, when the TATA box containing initiator region of the SV40 early promoter was substituted in the FR-α promoter, ER repression as well as derepression by antiestrogens was retained. This result suggests that the promoter specificity of ER response of FR-α resides entirely in the G/C-rich region of the P4 promoter.

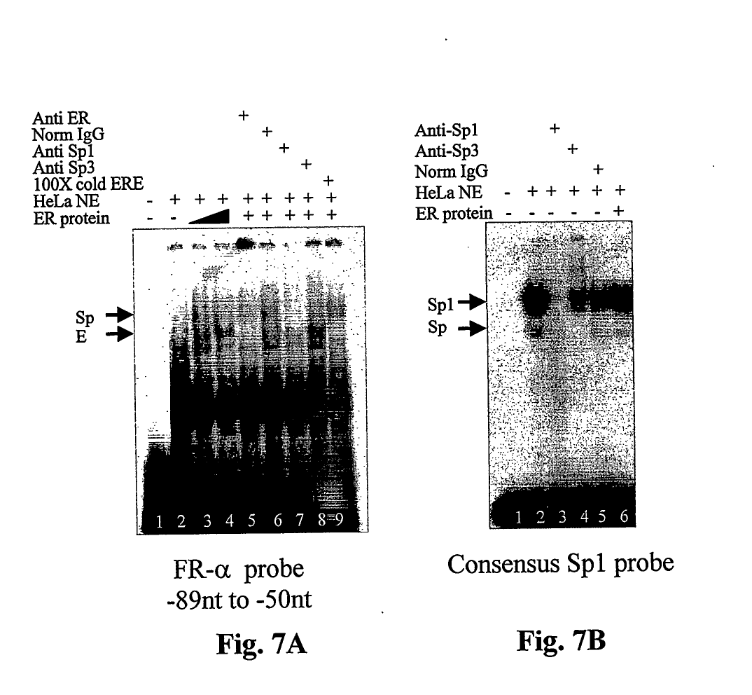

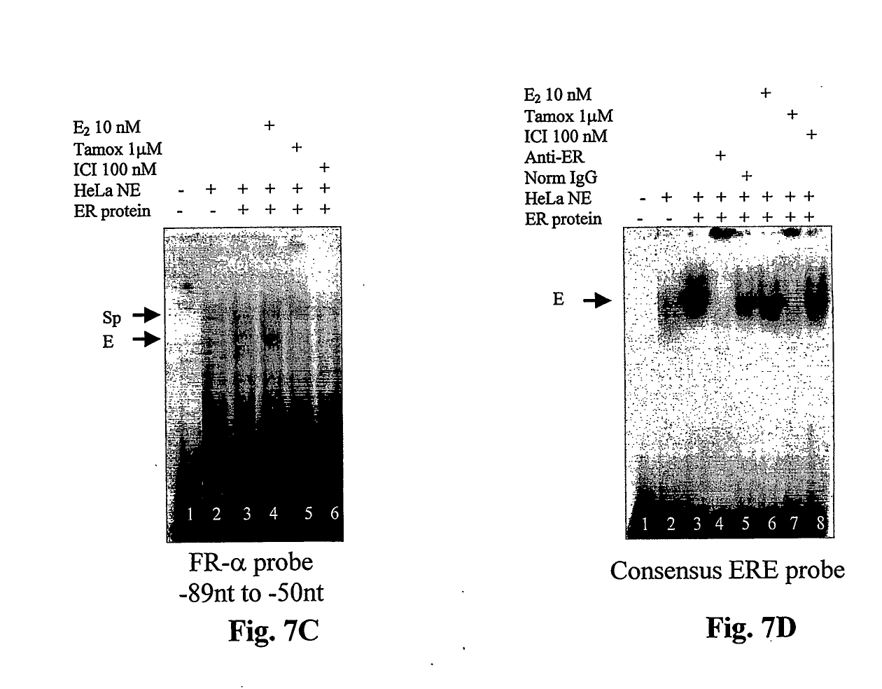

Identification of an ER interacting site in the P4 promoter of FR-α: The association of HeLa cell nuclear proteins and of ER with the P4 promoter was initially examined by electrophoretic gel mobility shift analysis (EMSA) of synthetic 40 bp DNA probes, covering the entire P4 promoter fragment including the G/C-rich and initiator regions; the EMSA probes were designed to have 20 bp overlaps. Only one of the overlapping probes (−89 nt to −50 nt) [Seq. ID No. 17] showed a shift that could be attributed to ER (FIG. 7). This probe contains one of the three Sp1 elements of the FR-1 promoter together with its flanking sequences. The ER associated shift was not observed when either a portion of the Sp1 element or the 3′ flanking sequence was deleted (results not shown) indicating a requirement for both the Sp1 binding and the flanking sequences. Antibodies to either Sp1 or ER completely blocked specific and distinct EMSA bands as indicated in FIG. 7A. Antibody to Sp3 (but not an isotype control) also blocked the Sp1 band presumably due to cross-reactivity with Sp1 as seen in FIG. 7B for a consensus (control) Sp1 element. The quantitative blocking of this band by the Sp1-specific antibody (FIG. 7A) as well as the different mobilities of binary DNA complexes of Sp1 and Sp3 (FIG. 7B) exclude the possibility that Sp3 may bind to the −89 nt to −50 nt sequence [Seq. ID No. 17] of the P4 promoter. ER, on the other hand, failed to show a shift with the consensus Sp1 element (FIG. 7B) demonstrating specificity for the FR-α probe. Further characterization of the EMSA band resulting from interaction of the −89 nt to −50 nt probe [Seq. ID No. 17] with ER is shown in FIGS. 7A and 7C. A 100-fold excess of an unlabelled probe containing a classical ERE sequence blocked the ER band in FIG. 7A confirming that the band is due to ER. E₂ increased the intensity of the ER band, whereas both ICI 182,780 and tamoxifen abolished it (FIG. 7C). In contrast, when a control probe containing a classical ERE sequence was used, tamoxifen, but not ICI 182,780 abolished the EMSA band due to ER (FIG. 7D). The above results demonstrate a direct interaction of ER with a specific G/C-rich region [Seq. ID No. 7] of the FR-α P4 promoter and show that this interaction is profoundly affected by ER ligands.

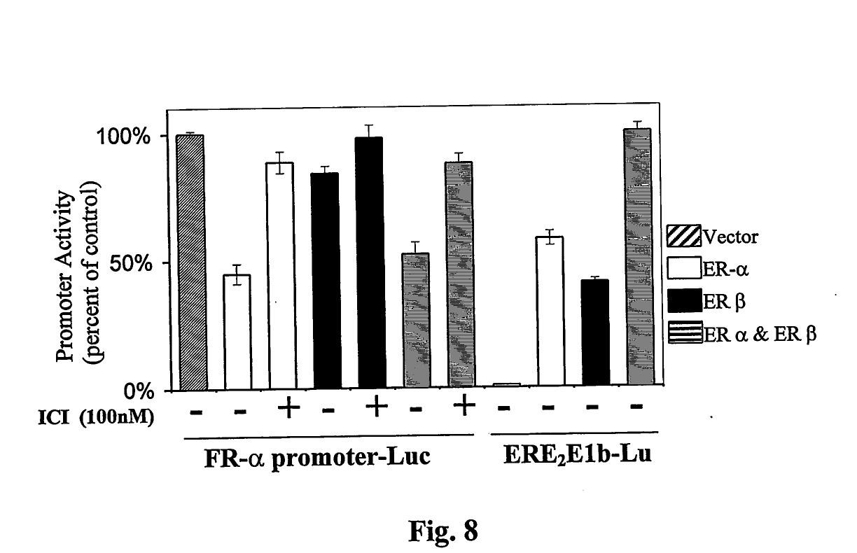

Effect of ERβ on FR-α Promoter activity: Since ER+ gynecological tumors are known to frequently co-express ERα and ERβ, the inventor herein tested whether ERβ may modulate FR-α similar to ERα or may otherwise counteract the effect of ERα. As seen in FIG. 8, ERβ caused a relatively modest repression of the FR-α promoter even though its full functionality was evident from its activation of the control ERE₂ E1b promoter. The modest repression of the FR-α promoter by ERβ was reversed by ICI 182,780 (FIG. 8). Co-transfection of ERβ and ERα, however, did not mitigate the ERα-mediated modulation of the FR-α promoter (FIG. 8) suggesting that ERβ will not competitively inhibit the effect of ERα on the FR-α promoter.

Effect of ER co-regulators on ER modulation of the FR-α promoter: Since the nature of the physiologic and pharmacologic effects of ER in a given tissue or cell type may be determined by the complement of co-activators and co-repressors expressed in those cells, it was undertaken to examine the effects of a variety of known ER co-regulators on ER-mediated regulation of the FR-α promoter. From Table 3 it is evident that whereas co-activators of the major SRC family (SRC-1, SRC-1E, TIF-2 and RAC3) as well as CBP enhanced ER activation of the control ERE₂E1b promoter to a variable degree, none of the co-activators appreciably altered either ER-mediated repression of the FR-α promoter or its activation by tamoxifen. In contrast, the classical co-repressor, SMRT, increased ER repression of the FR-α promoter (Table 3).

TABLE 3
Effect of coregulators on ER modulation of the FR-α promoter
I. Fold induction of Prompter Activity^{a}

| | | |
|---|---|---|
| ERE₂E1b-luc^{b} | FR-α promoter-Luc | |
| ER | ER | ER + Tamofaxifen^{c} |

II. Coregulator

| | | | |
|---|---|---|---|
| SCR-1 | 4.78 | 1.00 | 1.15 |
| SRC-1 | 4.92 | 0.99 | 0.81 |
| RAC-3 | 6.89 | 1.00 | 1.13 |
| TIF-2 | 4.67 | 0.97 | 1.12 |
| mCBP | 3.24 | 0.99 | 1.06 |
| SMRT | 0.63 | 0.35 | 0.67 |

^{a}Values indicate the fold induction of reporter luciferase activity due to a coregulator over the control luciferase activity in the absence or coregulator. The results represent at lest three independent experiments. Percent error <5.
^{b}Expression plasmids for the coregulators (100-300 ng) were cotransfected with 400 ng of each promoter construct and 25 ng ER plasmid in 1 × 10⁶ cells.
^{c}The cells were treated with 1 μM tamoxifen at the time of transfection

Discussion

The results of these examples demonstrate that both the endogenous FR-α gene and the plasmid FR-α promoter-reporter constructs are modulated by ER/ER ligands. The magnitude of the ER-ligand induced synthesis of FR-α was considerable in the chromosomal context of the endogenous FR-α gene. Antiestrogens, however, did not induce FR-α-expression in a variety of ER⁺/FR-α-negative cell lines indicating that they will not alter the tissue specificity of FR-α expression. ER ligands did not alter FR-α expression when the gene was driven by a constitutive (RSV) promoter (results not shown) excluding the possibility of post-transcriptional effects of ER on FR-α expression.

The examples herein were designed to address key mechanistic issues that pertain to the predictability of an in vivo tumor response of FR-α upregulation by antiestrogens. The close parallel in the ER and ER ligand dose response of repression of the FR-α promoter and the classical activation of the ERE₂E1b promoter support the finding that the ER ligands modulate FR-α in tumors at physiologic/pharmacologic levels of ER, estrogen and antiestrogens. In both promoters, transcriptional modulation by ER was observed even when the culture media was devoid of estrogenic molecules and the serum in the culture was previously treated with dextran-coated charcoal to deplete estrogen, even though the addition of E₂ did further increase the transcriptional effects of ER. Since ER activates promoters in a ligand-independent manner, depending on its phosphorylation state (3a, 4a) the above finding shows that unliganded ER is capable of repressing the FR-α promoter and that optimal repression is obtained in the presence of subnanomolar (physiologic) levels of estrogen. The short-term reversibility of the effect of tamoxifen but not ICI 182,780 on both the ERE₂E1b and FR-α promoters also supports a fundamental similarity between the two promoter responses in terms of the mode of action of the antiestrogens on ER in vivo, i.e., reversible alteration of the conformation of ER by tamoxifen (5a) and down-regulation of ER by ICI 182,780 (6a-8a). The time course of the antiestrogen response of the FR-α promoter was relatively rapid, consistent with the view that the FR-α gene is a direct target of ER action (discussed below).

Extensive investigations of ER have not revealed any DNA sequence element to which ER can bind directly besides the consensus ERE and its variants or ERE half-sites (9a). However, estrogen-ER complexes have been found to activate promoters through AP-1 or AP-1-like elements, an NFκB response element, G/C-rich (Sp1 binding) elements or other elements (10a-20a). Repression of promoters by E₂/ER is relatively less frequent and not as well characterized and may occur by direct interaction of ER with transcription factors such as NFκβ, Sp3 or GATA-1 (15a, 20a, 21a). The ER repression of the FR-α promoter was mapped to the proximal region of the P4 promoter, which lacks an ERE but contains an AP-1-like element and a cluster of G/C-rich elements. Mutational analysis excluded a role for the AP-1-like element but identified the G/C-rich region as the site of ER action. The list of genes activated by E₂ via an ER/Sp1 complex (at G/C-rich elements) is growing and includes E2F1, bcl-2, progesterone receptor, retinoic acid receptor α₁ cathepsin D, c-fos, IGF-binding protein 4, adenosine deaminase, thymidylate synthase, DNA polymerase α, telomerase, EGF receptor etc. (18a). There is a single reported example of promoter repression by E₂/ER at a G/C-rich element i.e., the vascular endothelial growth factor (VEGF) gene (20a), where the ER repression appears to be mediated by an ER/Sp3 complex, but a role for the remainder of the promoter context was not ruled out. The present examples of chimeras of the TATA-less FR-α promoter and the TATA box-dependent SV40 early promoter, show that the entire promoter specificity of E₂/ER repression of the FR-α promoter resides within the G/C-rich region of the FR-α gene. In the chimeric promoter in which the G/C-rich region of the FR-α promoter was replaced by the known Sp1 binding sequence of the SV40 promoter, E₂/ER activated the promoter, consistent with the action of the ER/Sp1 complex. Thus, the FR-α promoter-specificity for repression by ER must be determined by the specific sequence of the G/C-rich region of the P4 promoter. From EMSA, it appears that ER directly interacts with only one of the three Sp1 binding sites in the P4 promoter. The specificity for this site includes both the Sp1 binding element and its 3′ flanking sequence. In contrast to the VEGF gene promoter, Sp1 (but not Sp3) bound at this site. The formation of this ER complex occurred in the absence of E₂ but increased in the presence of E₂. This finding is consistent with the functional data on repression of the FR-α promoter by unliganded ER and its further repression when E₂ is present. Furthermore, both tamoxifen and ICI 182,780 prevented formation of the complex, offering a mechanistic explanation for the antagonistic effects of the ligands on ER repression of the FR-α gene. In contrast to the FR-α promoter element, ICI 182,780 did not prevent the binding of ER to the ERE sequence in vitro indicating a difference in the modes of interaction of ER with the two elements. Indeed, the unique manner in which ER associates with the FR-α promoter enables it to recruit transcriptional co-repressors rather than co-activators (discussed below).

Depending on the target gene and tissue, a variety of ER ligands, collectively known as selective ER modulators (SERMs), may act as agonists or antagonists of the transcriptional effects of estrogen by reversibly modulating the conformation of the receptor (5a). Contrary to SERMs (e.g., tamoxifen), pure antiestrogens, such as ICI 182,780 completely attenuate both ligand-dependent and ligand-independent functions of ER by multiple mechanisms in vivo. ICI 182,780 is known to impair ER dimerization, increase ER degradation and interfere with nuclear localization of the receptor (6a-8a). Consistent with the known actions of the antiestrogens on ER in vivo, the derepression of the FR-α promoter by tamoxifen but not ICI 182,780 was reversible in the short term. The ability of estrogen to activate a promoter and for a SERM to either promote or antagonize this activation is known to be determined by specific coregulator(s) (co-activators and co-repressors) recruited by ER which in turn is governed by the tissue-specific complement of the ER coregulators (3a, 5a, 9a, 22a). In E₂/ER-mediated gene repression, however, there is little information available on a potential role of coregulators, even though mutational analysis has provided indirect evidence to suggest a role for coregulators in gene repression by an ER/Ap1 complex (13a). None of the ER coactivators tested, including the major SRC family proteins, altered ER repression of the FR-α promoter or its derepression by tamoxifen; on the other hand, the ER co-repressor, SMRT, increased the repression. These findings support the view that FR-α gene repression by ER is an active process that involves recruitment of co-repressors but that the increase in FR-α promoter activity by antiestrogens may simply represent a passive process of derepression in which the antiestrogens disable ER. The results clearly indicate that the variable coregulator complement of a target tumor should not be a significant concern in extending the findings of antiestrogen modulation in cell lines to tumor tissues in vivo.

Most studies of estrogen action have focused on ERα since ERβ was discovered more recently (22a). The two ER types show differential expression as well as co-expression in various normal tissues but notably, they have been found to be co-expressed in epithelial cells in ovarian cancers (23a). ERα and ERβ may respond differently to ER ligands in a cell and target gene context dependent manner (10a, 17a) and they may even form functional heterodimers; ERβ can also inhibit ERα transcriptional activity (24a-26a). It has been demonstrated that both ERα and ERβ form similar complexes with Sp1, binding to its C-terminal region (27a). However, in a model promoter, activation by the ER/Sp1 complex required the AF-1 domain of ERα; ERβ was unable to activate the promoter due to a non-functional AF-1 domain (27a). The same study reported that when ERα and ERβ were co-expressed, ERβ inhibited promoter activation by ERα/Sp1. The above considerations raised the concern that in tumor cells expressing both ERα and ERβ, the latter may counteract ERα-dependent regulation of the FR-α gene. However, ERβ did mediate repression of the FR-α promoter, albeit to a modest extent and the promoter activity was restored by antiestrogens. More important, ERβ did not decrease the ERα-mediated repression of the FR-α promoter. This observation underscores potential differences in the ability of ERα vs. ERβ to associate with specific non-ERE elements.

Based on the foregoing results, it is believed that FR-α expression in ERα+ tumors is repressed by ER in the presence of physiologic levels of ER and estrogen and that pharmacologic doses of antiestrogens specifically and substantially increase the receptor levels independent of both ER-β expression and the cellular co-regulator complement. The short-term treatment with ER ligands for the specific purpose of temporarily elevating FR-α levels provides a beneficial therapeutic strategy since current clinical trials of SERMS as chemopreventatives (28a) involve protracted periods of treatment. The present invention establishes an especially useful protocol for FR-α modulation with ER ligands for imaging and treatment in major types of gynecological cancers and for the further development of gene repression by ER.

Studies of FR-α regulation by the estrogen receptor, the androgen receptor, the progesterone receptor and the glucocortocoid receptor: Among steroid receptors, the inventor has shown FR-α upregulation by ER, PR, AR and GR. Despite the frequent expression of the different steroid receptors in gynecological tumor subtypes that consistently express FR-α (for example, non-mucinous adenocarcinomas of the ovary and uterine endometrial adenocarcinoma), established tumor cell lines largely fail to mimic this co-expression pattern (with the exception of GR). Nevertheless, HeLa cells, which have been commonly used in studies of steroid receptors by transient transfection, are excellent model cells for studies of FR-α promoter regulation because the endogenous FR-α gene is active in these cells. In the following examples, the inventor has mostly used HeLa cells that have endogenous GR or recombinant HeLa cells that also express PR or ER. The HeLa cell transfection experiments were reproducible in other FR-α+ cell lines such as IGROV1 and SKOV3. T47D cells that express endogenous PR and GR were used. For the AR studies, HeLa cells were transfected with AR. Unless otherwise indicated, the FR-α promoter-luciferase reporter construct used in the following experiments includes the promoter fragment from −3394 nt to +33 nt [Seq. ID No. 6] which includes both the P1 and P4 promoters.

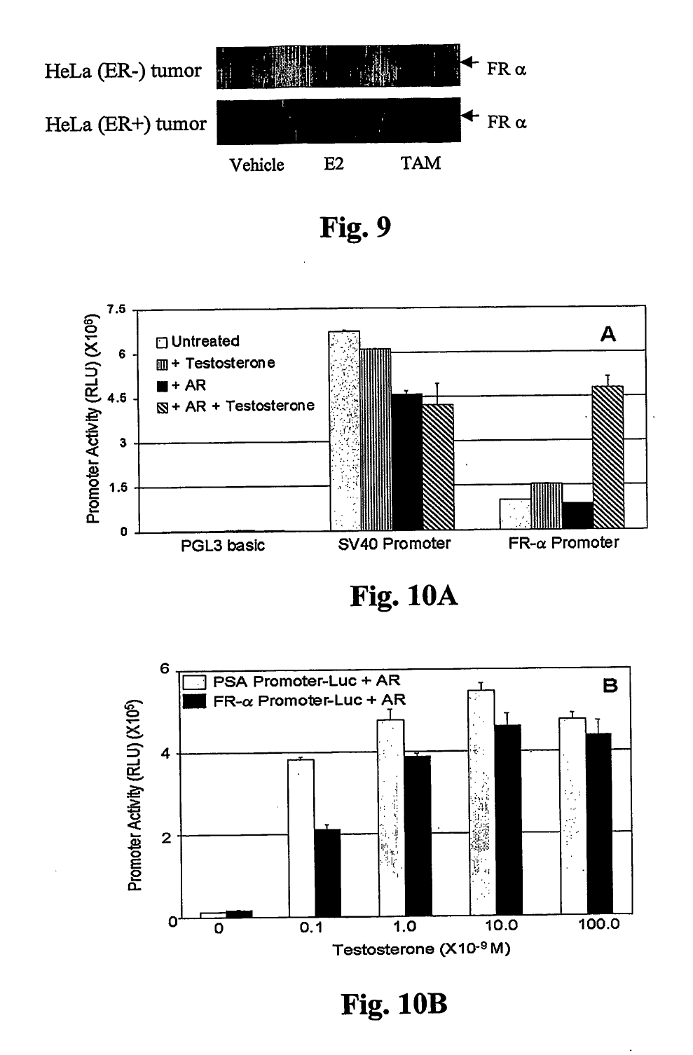

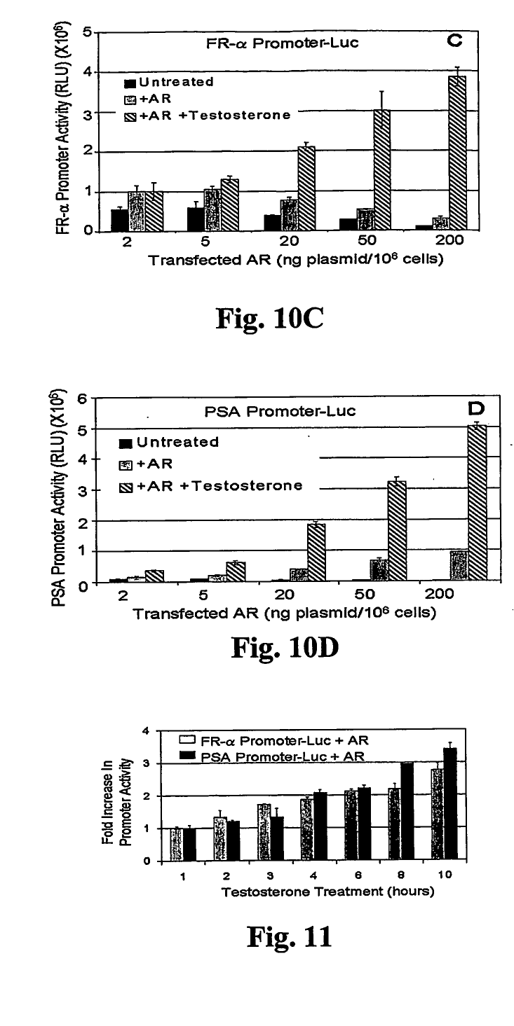

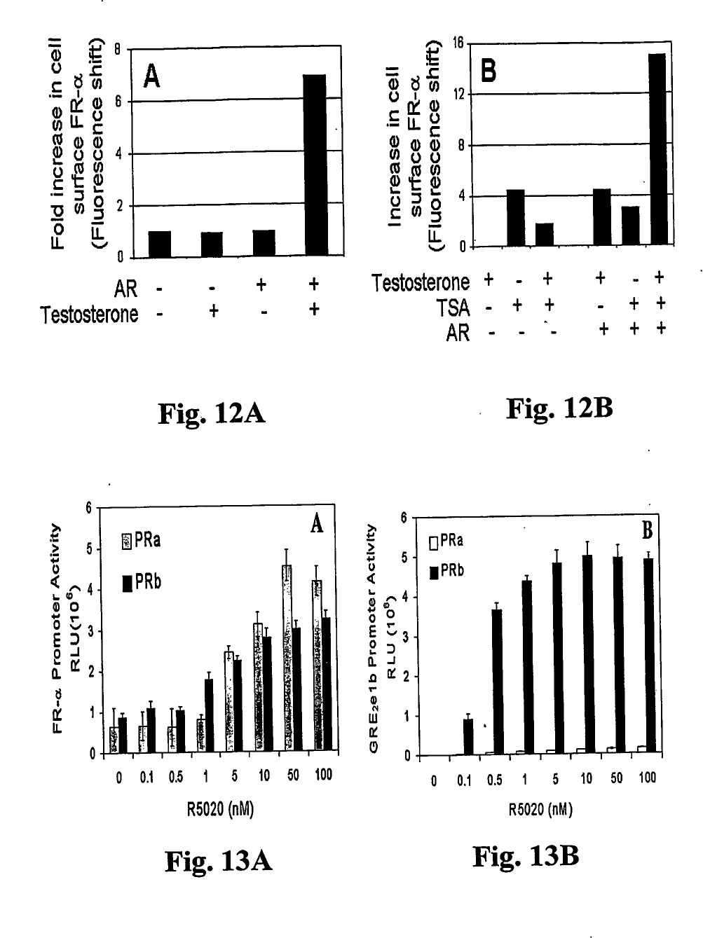

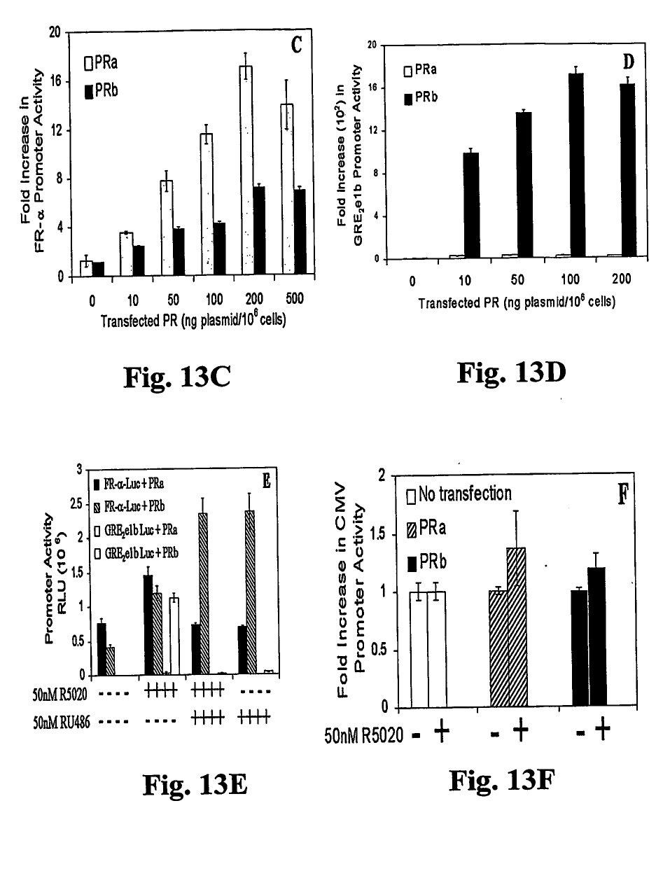

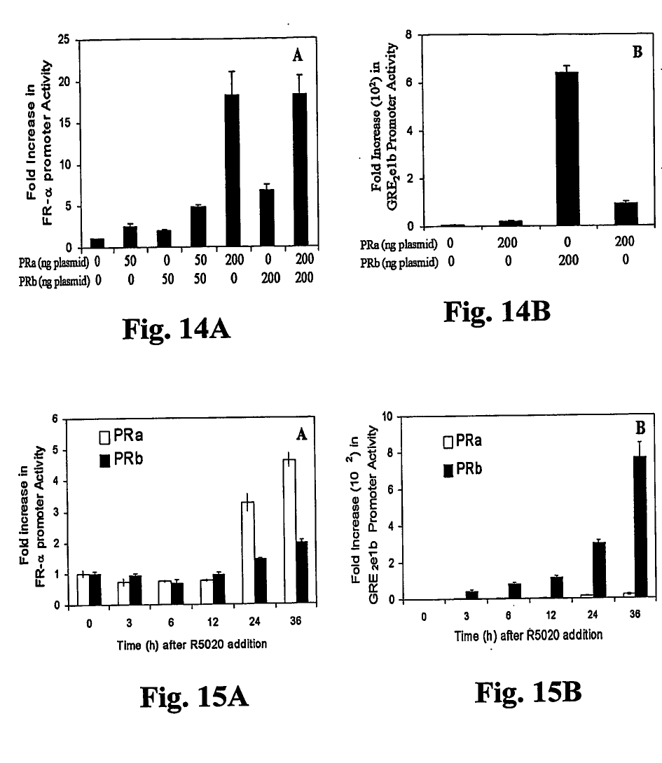

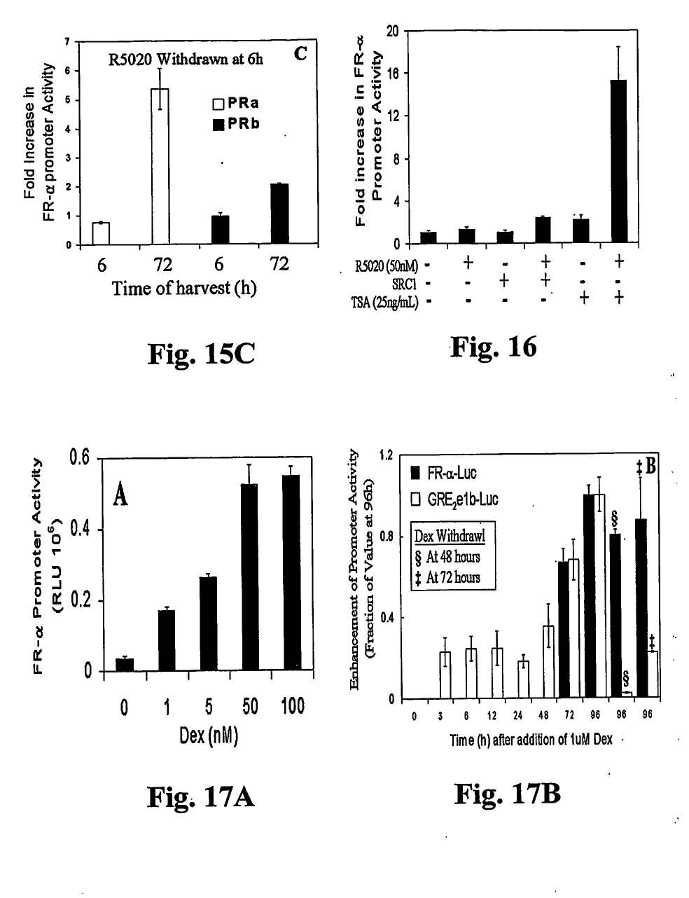

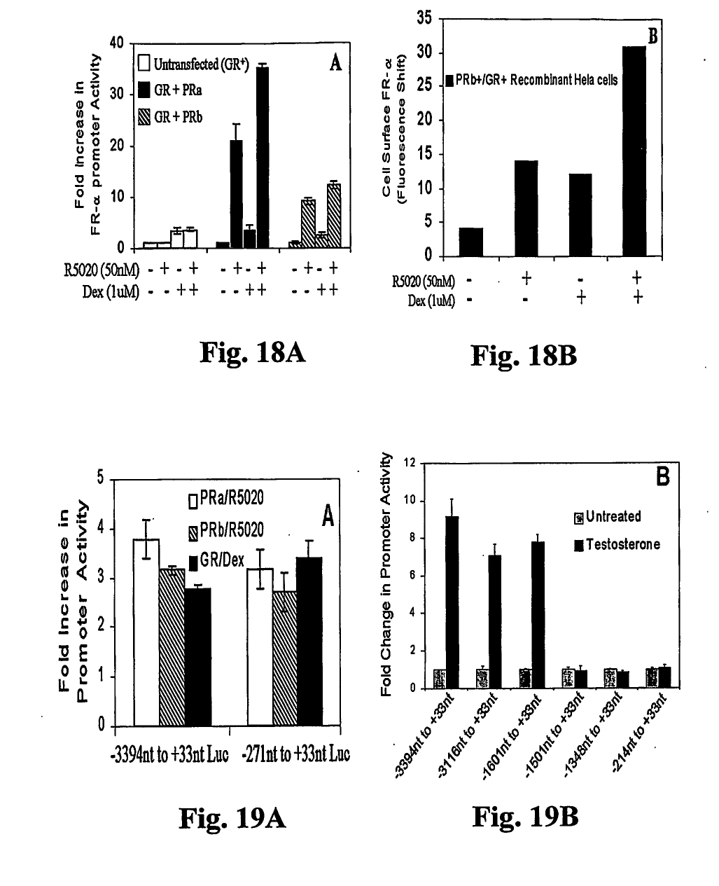

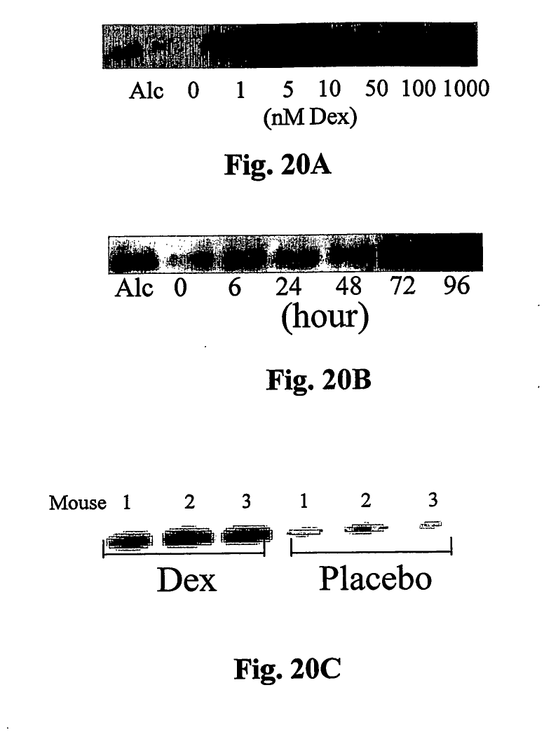

Upregulation of the FR-α Gene by Antiestrogen in Tumors in vivo: The negative regulation of FR-α by both the unliganded and estrogen bound ER and FR-α upregulation by antiestrogens has been described above. In FIG. 9, some initial representative unpublished data using recombinant ER+/FR-α+ HeLa cells or parental ER−/FR-α+ HeLa cells in SCID mouse tumor xenograft models is shown. Here, the (subcutaneous) tumor-bearing mice were administered (sc) either vehicle or tamoxifen for 4 days prior to sacrifice and analysis of FR-α expression by western blot. A set of typical data, shown in FIG. 9, shows a significant increase in FR-α expression due to tamoxifen treatment in vivo in the ER+ but not in the ER⁻ HeLa cell tumors. This demonstrates the potential in vivo relevance of in vitro studies of FR-α regulation by steroid receptors.
Regulation of FR-α by the Androgen Receptor: Testosterone and AR Dose response and Promoter Specificity: In HeLa cells transiently co-transfected with AR and the FR-α promoter-luciferase construct, testosterone (10 nM) increased the promoter activity in an testosterone/AR-dependent manner (FIG. 10A) but not in an SV40 promoter-luciferase construct (FIG. 10A). The activation of the FR-α promoter occurred in a testosterone dose-dependent manner and maximum activation occurred between 1 nM and 10 nM testosterone (FIG. 10B). The testosterone dose response paralleled that observed for the activation of the promoter for prostate-specific antigen (PSA), which contains a classical androgen response element (FIG. 10B). The AR dose response (in terms of ng of transfected AR plasmid/10⁶ cells) for activation of the FR-α promoter by testosterone (10 nM) (FIG. 10C) also paralleled that of the PSA promoter (FIG. 10D). These results demonstrate specific AR-dependent activation of the FR-α promoter by testosterone.
Time Course of Activation of the FR-α Promoter by Testosterone: The time course was initiated 48 h after co-transfection of HeLa cells with AR and either FR-α promoter-luciferase or PSA promoter-luciferase in order to ensure that the level of AR did not change appreciably during the time course (western blot data for AR expression, not shown). As seen in FIG. 11, in both promoters, activation was observed at 2 h and progressed at a similar rate. In a separate experiment (data not shown), the inventor failed to observe a delayed stimulation of the FR-α promoter by testosterone/AR as he did for R5020/PR and for dexamethasone/GR (discussed in later sections). These results suggest that the FR-α promoter could possibly (but not necessarily) be a direct target for AR action.
Upregulation of Endogenous FR-α by Testosterone/AR and Potentiation of the AR action by TSA: In FIG. 12A, untransfected HeLa cells or cells transfected with AR expression plasmid were treated with either vehicle alone or with testosterone (10 nM) for 3 days. The expression of endogenous FR was measured by quantifying the binding of a fluorescein conjugate of folic acid to the cell surface by flow cytometry. There was a testosterone/AR-dependent increase in fluorescence of the major peak (presumably representing the transfected population of cells) of approximately 7-fold (FIG. 12A). This increase was further enhanced (˜4-fold) by treating the cells with 50 ng/ml TSA (FIG. 12B). The data clearly indicates that the enhancement of FR-α promoter activity by testosterone/AR, observed above, is reflected in the regulation of the endogenous FR-α gene and that inhibition of HDAC can potentiate this effect. Testosterone did not induce FR (α or β) expression in any of a variety of cell types tested that are FR-negative (results not shown).
Regulation of FR-α Transcription by the Progesterone Receptors a and b: Ligand and Receptor Dose Response and Promoter-Specificity: FIG. 13A shows that in a transient transfection system, the potent PR agonist R5020 (progestin) increases FR-α promoter-luciferase reporter activity in a dose-dependent manner, with the maximal effects at 50 nM for PRa and 10 nM for PRb; the maximal activation produced by PRa was greater than that produced by PRb. In contrast, in a control promoter (GRE₂e1b) containing classical hormone (progesterone/glucocorticoid) response elements (GRE), the activity of PRb was much greater (nearly two orders of magnitude) than that of PRa and the maximal activation occurred at ˜1 nM (FIG. 13B). The relative PR dose response (in terms of ng PR plasmid DNA/10⁶ cells transfected) was in the range of 10 ng-200 ng for both PRa and PRb for the FR-α promoter (FIG. 13C) whereas maximum activation, produced by PRb on the GRE₂e1b-promoter was at 10 ng plasmid (FIG. 13D). The PR antagonist, RU486, inhibited transactivation of the GRE₂e1b promoter by both PRa and PRb in the presence of R5020 (FIG. 13E) Ru486 also inhibited the action of PRa/R5020 on the FR-α promoter but not that of PRb/R5020; on the other hand, PRb/Ru486 effectively activated the FR-α promoter (FIG. 13E). Neither PRa nor PRb significantly altered the activity of the CMV promoter in the presence of R5020 (50 nM) (FIG. 13F), indicating promoter specificity/selectivity under these experimental conditions. The above results demonstrate specific ligand and receptor-mediated activation of the FR-1 promoter by PR and show that it differs from a classical PR target promoter in terms of ligand and receptor subtype specificities and the much higher progestin dose required for substantial activation of the FR-α promoter.
Combined Effect of PRa and PRb: PRa and PRb are frequently co-expressed in vivo. When co-expressed at sub-optimal levels, PRa and PRb produced additive enhancement of the FR-α promoter activity (in the presence of 50 nM R5020) but at optimal levels, their combined degree of activation corresponded to that of PRa (the higher value) (FIG. 14A). In contrast, in the GRE₂e1b promoter, PRa strikingly inhibited the activation by PRb (FIG. 14B); this inhibition is presumably due to effective competition for GRE by PRa, which as seen above is much less transcriptionally active than PRb. These results show mechanistic differences in PR action on FR-α vs. the GRE-driven promoter. They also show that PRa and PRb work together to modulate FR-α in tumor tissues in which they are co-expressed.
Time Course and Reversibility of PR Effects: For the time course experiments in FIG. 8, R5020 (50 nM) was introduced 48 h after transfection with PR to ensure that PR levels did not change appreciably during the time course (western blot data for PR, not shown). In FIG. 15A, activation of the FR-α promoter began at 12 h-24 h with both PRa and PRb. In contrast, for the GRE₂e1b-promoter, activation was observed at ≦3 h with both PRa and PRb (FIG. 15B). To understand this apparent delayed response of the FR-α promoter, an experiment was designed in which the transfected cells were treated with R5020 for only 6 h and then either immediately harvested or washed free of the agonist and incubated for a further 66 h before harvesting (FIG. 15C). The absence of a significant amount of residual R5020 after the wash was ensured in a separate experiment in which R5020-treated and washed cells were transfected with a ligand-sensitive promoter and transactivation measured (data not shown). As seen in FIG. 15C, even though, as expected from the previous time course, there was no promoter activation at the time R5020 was withdrawn (i.e., 1-fold activation), a substantial amount of promoter activation occurred during a later period. This result strongly suggests that the FR-α gene may not be a direct target for PR action and that the promoter may be modulated by the product(s) of a specific upstream gene(s) target of PR. These examples thus identify the putative FR-α promoter-selective/specific transcription factor(s) that mediate this effect.
Co-activator Limitation and Potentiation by TSA of Endogenous PR action in T47D Cells: T47D breast carcinoma cells express endogenous PR. Treatment of these cells with R5020 (50 nM) did not activate transfected FR-α promoter-luciferase (i.e., 1-fold increase) (FIG. 16). However, co-transfection with expression plasmid for SRC1, a NR co-activator, resulted in activation of the FR-α promoter by R5020 (FIG. 16). FIG. 16 also shows that activation of the FR-α promoter in T47D cells by R5020 was greatly potentiated by treatment of the cells with the HDAC inhibitor, TSA (25 ng/ml). TSA and/or R5020 did not detectably alter PR expression under these conditions (western blot data not shown). These results illustrate that in certain cells at least one of the reasons for the inability of PR to mediate enhancement of FR-α promoter activity appears to be a limitation in co-activator availability and that in these cells a HDAC inhibitor (TSA) can substantially potentiate the activity of PR.
Up-regulation of Endogenous FR-α in Recombinant PR-positive HeLa Cells: Owing to the delayed effect of PR on FR-α promoter activity, stable expression of PR is required in order to allow a longer exposure to R5020 to accumulate a significant fold increase of endogenous FR-α expression. The inventor has produced stable recombinant HeLa cells expressing PRb. These cells were treated for 4 days with either R5020 (50 nM) or vehicle alone and the change in FR-α expressed on the cell surface was measured by flow cytometry using a fluorescein-conjugated folic acid probe (FIG. 18B). (The additional data in FIG. 18B is discussed below together with the GR studies). R5020/PRb significantly upregulated the endogenous FR-α protein expression even though, as noted above, PRb is less effective in enhancing FR-α promoter activity compared with PRa. R5020 did not induce FR (α or β) expression in a variety of PR-positive/FR-negative cells (data not shown).
Enhancement of FR-α Transcription by Dexamethasone/GR: Since GR is commonly expressed in tissues and cell lines, in the following experiments on the effects of the GR agonist, dexamethasone (Dex), the endogenous GR in HeLa cells was utilized. It may be noted that although the closely related MR binds endogenous glucocorticoids with a higher affinity than GR, Dex acts primarily through GR (237); moreover, since as mentioned above, MR has restricted tissue expression (in tissues involved in Na⁺ reabsorption), the Dex effects on FR-α transcription are presumed to be mediated by GR.
Dex Dose Response, Time Course and Reversibility: Dex produced optimal enhancement of transfected FR-α promoter-luciferase activity in HeLa cells, at a concentration of 50 nM (FIG. 17A). The time course of Dex activation of the GRE₂e1b promoter showed activation at ≦3 h (FIG. 10B). In contrast, enhancement of the FR-α promoter activity began only after 48 h (FIG. 17B). Furthermore, withdrawal of Dex at 48 h or 72 h did not appreciably decrease the FR-α promoter activity compared to the value after a 96 h treatment (FIG. 17B); in contrast, under similar conditions the activity of the GRE₂e1b promoter declined dramatically (FIG. 17B). These results indicate that the Dex enhancement of the FR-α promoter is likely mediated indirectly through the action(s) of upstream target gene(s) of Dex and that the mode of action of Dex on the FR-α promoter is likely fundamentally different from its activation of a GRE-driven promoter.
Potentiation of Dex activity in T47D cells by TSA: Similar to the observation above for R5020/PR, in FR-α-promoter-luciferase transfected T47D cells, Dex alone was unable to significantly alter the promoter activity; however, in the presence of TSA (25 ng/ml), Dex enhanced the promoter activity, similar to R5020 (results not shown).
Combined Enhancement of the FR-α Promoter Activity and Endogenous FR-1 Expression by Dex and R5020: In FIG. 18A, the GR+ HeLa cells were co-transfected with FR-α promoter-luciferase and plasmid expressing either PRa or PRb. As shown in FIG. 18A, in these cells, the combined enhancement of Dex and R5020 was greater than those of either ligand alone and in the case of PRa transfected cells, the increase was greater than additive. (Please note that the Dex effect was independent of PR). The increase in cell surface expression of the endogenous FR-α in stable recombinant HeLa cells expressing PRb was determined by flow cytometry (as described above) (FIG. 18B); the increase in FR-α in the presence of both Dex and R5020 was greater than the sum of the values for the individual ligands. These results suggest that GR and PR positively regulate the FR-α gene by distinct mechanisms which may synergistically contribute to FR-1 expression.
Mapping the Sites of Action of R5020/PR, Testosterone/AR and Dex/GR in the FR-α Promoter: The full-length FR-α promoter (containing both P1 and P4 promoters) (−3394 nt to +33 nt) [Seq. ID No. 6] and shorter 5′ deleted fragments of it were transfected into HeLa cells. The cells were co-transfected with PRa or PRb and treated with R5020 or treated with Dex without co-transfection. The shortest promoter fragment tested contains the P4 promoter and flanking regions (−271 nt to +33 nt) [Seq. ID No. 1], with the transcription initiation site of the P4 promoter designated the +1 nt position). This fragment retained responsiveness to R5020/PRa, R5020/PRb and Dex (FIG. 19A). This promoter fragment contains three Sp1 elements [Seq. ID No. 3] [Seq. ID No. 4] [Seq. ID No. 5] and one Ap1 element [Seq. ID No. 2]. ER directly associates with the G/C-rich and flanking sequence of one of the Sp1 sites. However, based on preceding data, it is quite possible that PR and GR interact with this region by an indirect (secondary) mechanism. By co-transfecting FR-α promoter-luciferase constructs with 5′ end deletions and AR plasmid into HeLa cells and measuring promoter activity, the inventor mapped the testosterone/AR-responsive region to a stretch of 100 bp (−1601 nt to −1501 nt) in the FR-α promoter [Seq. ID No. 18]. (FIG. 19B). This region lies downstream of the P1 promoter and 1500 bp upstream of the transcription initiation site of the P4 promoter. Since the P1 promoter was deleted in the shortest testosterone responsive construct in FIG. 19B, it is likely that the mapped region contains an enhancer for the P4 promoter. Interestingly, this sequence does not contain any known site for AR interaction, suggesting that it may contain a novel site of (direct or indirect) action of AR.
Upregulation of Endogenous FR-α by Dex In Vitro and In Vivo: The data in FIGS. 20A, B and C illustrates that consistent with the effect of Dex on the FR-α promoter activity discussed above, Dex strikingly upregulated the expression of endogenous FR-α in HeLa cells in vitro and in mouse xenografts of HeLa cell tumors in vivo. The Dex concentration dependence (FIG. 20A) and time course of the response to Dex (FIG. 20), were consistent with those for Dex-induced upregulation of FR-α promoter activity. The striking increase in FR-α expression observed in the tumor xenograft model (FIG. 20C) in Dex treated mice compared to the placebo group, demonstrates that Dex is able to induce FR-α expression in tumors in the physiologic milieu.

The foregoing examples clearly demonstrate a profound influence of steroid receptors on the expression of FR-o. The results show that combinations of steroid receptor ligands and a HDAC inhibitor can produce optimal enhancement of FR-α expression at the transcriptional level. The clinical implications of the above findings in FR-α-targeted diagnostics and therapeutics are compelling.

### Other Embodiments

The present invention is not to be limited in scope by the specific embodiments described that are intended as single illustrations of individual aspects of the invention and functionally equivalent methods and components are within the scope of the invention, in addition to those shown and described herein will become apparent to those skilled in the art from the foregoing description and accompanying figures. Such modifications are intended to fall within the scope of the appended claims.

The following references and any other references mentioned herein, to the extent that they provide exemplary procedural or other details supplementary to these set forth here, are specifically incorporated herein by reference. Further, all references cited herein, including journal articles, patents, and databases are expressly incorporated by reference.
1. Antony, A. C. (1996) Folate receptors. Ann. Rev. Nut. 16:501-521.
2. Antony, A. C. (1992) The biological chemistry of folate receptors. Blood 79:2807-2820.
3. Antony, A. C., Utley, C., VanHorne, K. C., and Kolehouse, J. F. (1981) Isolation and characterization of a folate receptor from human placenta. J. Biol. Chem. 256:9684-9692.
4. Suleiman, S. A. and Spector, R. (1981) Purification and characterization of a folate binding protein from porcine choroid plexus. Arch. Biochem. Biophys. 208:87-94.
5. Selhub, J. and Franklin, W. A. (1984) The folate-binding protein of rat kidney. Purification, properties, and cellular distribution. J. Biol. Chem. 259:6601-6606.
6. Selhub, J., Gay, A. G., and Rosenberg, I. H. (1979) Folate binding activity in epithelial brush border membrane. A system for investigating membrane associated folate binding proteins. In: Chemistry and Biology of Pteridines, Elsevier, N.Y., pp. 593-596.
7. Elwood, P. C., Kane, M. A., Portillo, R. M., and Kolhouse, J. F. (1986) The isolation characterization, and comparison of the membrane-associated and soluble folate-binding proteins from human KB cells. J. Biol. Chem. 261:15416-15423.
8. Antony, A. C., Utley, C. S., Marcell, P. D., and Kolhouse, J. F. (1982) Isolation, characterization, and comparison of the solubilized particulate and soluble folate binding proteins from human milk. J. Biol. Chem. 257:10081-10089.
9. Kamen, B. A. and Caston, J. D. (1975) Purification of folate binding factor in normal umbilical cord serum. Proc. Natl. Acad. Sci. USA 72:4261-4264.
10. Hansen, S. I., Holm, J., and Lyngbye, J. (1985) A high-affinity folate binding protein in human cerebrospinal fluid. Acta Neurol. Scand. 71:133-135.
11. Holm, J., Hansen, S. I., and Hoier-Madsen, M. (1990) A high affinity folate binding protein in human amniotic fluid. Radioligand binding characteristics, immunological properties and molecular size. Bioscience Reports 10:79-85.
12. Hansen, S. I., Holm, J., and Hoier-Madsen, M. (1989) A high-affinity folate binding protein in human urine. Radioligand binding characteristics, immunological properties and molecular size. Bioscience Reports 9:93-97.
13. da Costa, M. and Sharon, M. (1980) The synthesis of folate-binding protein in lymphocytes during transformation. Br. J. Haematol. 46:575-579.
14. Luhrs, C. A. and Slomiany, B. L. (1989) A human membrane-associated folate binding protein is anchored by a glycosylphosphatidylinositol tail. J. Biol. Chem. 264:21446-21449.
15. Kane, M. A., Elwood, P. C., Portillo, R. M., Antony, A. C., and Kolhouse, J. F. (1986) The interrelationship of the soluble and membrane-associated folate-binding proteins in human KB cells. J. Biol. Chem. 261:15625-15631.
16. Verma, R. S., Gullapalli, S., and Antony, A. C. (1992) Evidence that the hydrophobicity of isolated, in situ, and de novo-synthesized native human placental folate receptors is a function of glycosyl-phosphatidylinositol anchoring to membranes. J. Biol. Chem. 267:4119-4127.
17. Elwood, P. C. (1989) Molecular cloning and characterization of the human folate-binding protein cDNA from placenta and malignant tissue culture (KB) cells. J. Biol. Chem. 264:14893-14901.
18. Lacey, S. W., Sanders, J. M., Rothberg, K. G., Anderson, R. G., and Kamen, B. A. (1989) Complementary DNA for the folate binding protein correctly predicts anchoring to the membrane by glycosyl-phosphatidylinositol. J. Clin. Invest. 84:715-720.
19. Ratnam, M., Marquardt, H., Duhring, J. L., and Freisheim, J. H. (1989) Homologous membrane folate binding proteins in human placenta: cloning and sequence of a cDNA. Biochemistry 28:8249-8254.
20. Shen, F., Ross, J. F., Wang, X., and Ratnam, M. (1994) Identification of a novel folate receptor, a truncated receptor and receptor type β in hematopoietic cells: cDNA cloning, expression, immunoreactivity and tissue specificity. Biochemistry 33:1209-1215.
21. Shen, F., Wu, M., Ross, J. F., Miller, D., and Ratnam, M. (1995) Folate receptor type γ is primarily a secretory protein due to lack of an efficient signal for glycosylphosphatidylinositol modification: protein characterization and cell type specificity. Biochemistry 34:5660-5665.
22. Yan, W. and Ratnam, M. (1995) Preferred sites of glycosylphosphatidylinositol modification in folate receptors and constraints in the primary structure of the hydrophobic portion of the signal. Biochemistry 34:14594-14600.
23. Wang, H., Ross, J. F. and Ratnam, M. (1998) Structure and regulation of a polymorphic gene encoding folate receptor type γ/γ′. Nucl. Acids Res. 26:2312-2142.
24. Turek, J. J., Leamon, C. P. and Low, P. S. (1993) Endocytosis of folate-protein conjugates: ultrastructural localization in KB cells. J. Cell Sci. 106:423-430.
25. Leamon, C. P. and Low, P. S. (1991) Delivery of macromolecules into living cells: a method that exploits folate receptor endocytosis. Proc. Natl. Acad. Sci. USA 88:5572-5576.
26. Wu, M., Fan, J., Gunning, W., and Ratnam, M. (1997) Clustering of GPI-anchored folate receptor independent of both cross-linking and association with caveolin. J. Membrane Biol. 159:137-147.
27. Wang, X., Shen, F., Freisheim, J. H., Gentry, L. E., and Ratnam, M. (1992) Differential stereospecificities and affinities of folate receptor isoforms for folate compounds and antifolates. Biochem. Pharmacol. 44:1898-1901.
28. Shen, F., Zheng, X., Wang, J., and Ratnam, M. (1997) Identification of amino acid residues that determine the differential ligand specificities of folate receptors α and β. Biochemistry 36:6157-6163.
29. Maziarz, K. M., Monaco, H. L., Shen, F., and Ratnam; M. (1999) Complete mapping of divergent amino acids responsible for differential ligand binding of folate receptors α and β. J. Biol. Chem. 274:11086-11091.
30. Weitman, S. D., Lark, R. H., Coney, L. R., Fort, D. W., Frasca, V., Vincent, R., Zurawski, J., and Kamen, B. A. (1992) Distribution of the folate receptor GP38 in normal and malignant cell lines and tissues. Cancer Res. 52:3396-3401.
31. Veggian, R., Fasolato, S., Menard, S., Minucci, D., Pizzetti, P., Regazzoni, M., Tagliabue, E., and Colnaghi, M. I. (1989) Immunohistochemical reactivity of a monoclonal antibody prepared against human ovarian carcinoma on normal and pathological female genital tissues. Tumori 75:510-513.
32. Buist, M. R., Molthoff, C. F. M., Kenemans, P., and Meijer, C. J. L. M. (1995) Distribution of OV-TL3 and MOv18 in normal and malignant ovarian tissue. J. Clin. Pathol. 48:631-438.
33. Wu, M., Gunning, W., and Ratnam, M. (1999) Expression of folate receptor type α in relation to cell type, malignancy and differentiation in ovary, uterus and cervix. Cancer Epidemiol. Biomarkers & Prevention 8:775-782.
34. Campbell, I. G., Jones, T. A., Foulkes, W. D., and Trowsdale, J. (1991) Folate binding protein is a marker for ovarian cancer. Cancer Res. 51:5329-5338.
35. Garin-Chesa, P., Campbell, I., Saigo, P. E., Lewis, J. L., Jr., Old., L. J., and Rettig, W. J. (1993) Trophoblast and ovarian cancer antigen LK26. Sensitivity and specificity in immunopathology and molecular identification as a folate-binding protein. Am. J. Pathol., 142:557-567.
36. Ross, J. F., Chaudhuri, P. K., and Ratnam, M. (1994) Differential regulation of folate receptor isoforms in normal and malignant tissues in vivo and in established cell lines: physiologic and clinical implications. Cancer 73:2432-2443.
37. Weitman, S. D., Frazier, K. M., and Kamen, B. A. (1994) The folate receptor in central nervous system malignancies of childhood. J. Neurooncol., 21:107-112, 1994.
38. Ross, J. F., Wang, H., Behm, F. G., et al. (1999) Folate receptor type β is a neutrophilic lineage marker and is differentially expressed in myeloid leukemia. Cancer 85:348-357.
39. Pan, X. Q., Zheng, X., Shi, G., Wang, H., Ratnam, M., and Lee, R. J. (2002) Strategy for the treatment of acute myelogenous leukemia based on folate receptor beta-targeted liposomal doxorubicin combined with receptor induction using all-trans retinoic acid. Blood, 100:594-602.
40. Nakashima-Matsushita, N., Homma, T., Yu, S., Takemasa, M., Sunahana, N., Nakamura, T., Tsukano, M., Ratnam, M., and Matsuyama, T. (1999) Selective expression of folate receptor β and its possible role in methotrexate transport in synovial macrophages from patients with rheumatoid arthritis. Arthritis and Rheumatism 42:1609-1616.
41. Toffoli, G., Cernigo, C., Russo, A., Galls, A., Bagnoli, M., and Boiocchi, M. (1997) Overexpression of folate binding protein in ovarian cancers. Int. J. Cancer 74:193-198.
42. da Costa, M., Rothenberg, S. P., Sadasivan, E., Regec, A., and Qian, L. (2000) Am. J. Physiol. Cell. Physiol. 278:C812-C821.
43. Piedrahita, J. A., Oetama, B., Bennett, G. D., Van Waes, J., Kamen, B. A., Richardson, J., Lacey, S. W., Anderson, R. G., and Finnell, R. H. (1999) Mice lacking the folic acid-binding protein Fo1bp1 are defective in early embryonic development. Nat. Genet. 23:228-232.
44. Wlodarczyk, B., Spiegelstein O., Gelineau-van Waes, J., Vorce R. L., Lu, X., Le, C. X, Finell, R. H. (2001) Arsenic-induced congenital malformations in genetically susceptible folate binding protein-2 knockout mice. Toxicol. Appl. Pharmacol. 177:238-246.
45. Leamon, C. P. and Low, P. S. (2001) Folate-mediated targeting: from diagnostics to drug and gene delivery. Drug Discovery Today 6:44-51.
46. Lu, Y., Low, P. S. (2002) Folate-mediated delivery of macromolecular anticancer therapeutic agents. Adv. Drug Deliv. Rev. 54:675-693.
47. Drummond, D. C., Hong, K., Park, J. W., Benz, C. C., Kirpotin, D. B. (2000) Liposome targeting to tumors using vitamin and growth factor receptors. Vitam. Horm. 60:285-332.
48. Sudimack, J. and Lee, R. J. (2000) Targeted drug delivery via the folate receptor. Adv. Drug Deliv. Rev. 41:147-162.
49. Ward, C. M. (2000) Folate-targeted non-viral DNA vectors for cancer gene therapy. Curr. Opin. Mol. Ther. 2:182-187.
50. Leamon, C. P. and Low, P. S. (1994) Selective targeting of malignant cells with cytotoxin-folate conjugates. J. Drug Targeting 2:101-112.
51. Ladino, C. A., Chari, R. V., Bourret, L. A., Kedersha, N. L., and Goldmacher, V. S. (1997) Folate-maytansinoids. Target-selective drugs of low molecular weight. Int. J. Cancer 73:859-864.
52. Atkinson, S. F., Bettinger, T., Seymour, L. W., Behr, J. P., Wart, C. M. (2001) Conjugation of folate via gelonin carbohydrate residues retains ribosomal-inactivating properties of the toxin and permits targeting to folate receptor positive cells. J. Biol. Chem. 276:27930-27935.
53. Mathias, C. J., Wang, S., Low, P. S., Waters, D. J., and Green, M. A. (1999) Receptor-mediated targeting of 67Ga-deferoxamine-folate to folate-receptor-positive human KB tumor xenografts. Nucl. Med. Biol. 26:23-25.
54. Mathias, C. J., Hubers, D., Low, P. S., and Green, M. A. (2000) Synthesis of [(99m)Tc]DTPA-folate and its evaluation as a folate-receptor-targeted radiopharmaceutical. Bioconjug. Chem. 11:253-257.
55. Andersson, H., Lindegren, S., Back, T., Jacobsson, L., Leser, G., and Horvath, G. (2000) Radioimmunotherapy of nude mice with intraperitoneally growing ovarian cancer xenograft utilizing 211At-labelled monoclonal antibody MOv18. Anticancer Res. 20:459-462.
56. Visser, G. W., Klok, R. P., Gebbinck, J. W., ter Linden, T., van Dongen, G. A., Molthoff, C. F. (2001) Optimal quality (131)I-monoclonal antibodies on high-dose labeling in a large reaction volume and temporarily coating the antibody with IODO-GEN. J. Nucl. Med. 42:509-519.
57. Leamon, C. P., Parker, M. A., Vlahov, I. R., Xu, L. C., Reddy, J. A., Vetzel, M., Douglas, N. (2002) Synthesis and biological evaluation of EC20: A new folate-derived (99m)Tc-based radiopharmaceutical. Bioconjug. Chem. 6:1200-1210.
58. Konda, S. D., Wang, S., Brechbiel, M., Weiner, E. C. (2002) Biodistribution of a 153 Gd-folate dendrimer, generation=4, in mice with folate-receptor positive and negative ovarian tumor xenografts. Invest. Radiol. 4:199-204.
59. Lee, R. J. and Low, P. S. (1994) Delivery of liposomes into cultured KB cells via folate receptor-mediated endocytosis. J. Biol. Chem. 269:3198-3204.
60. Wang, S., Lee, R. J., Cauchon, G., Goreustein, D. G., and Low, P. S. (1995) Delivery of antisense oligo deoxyribonucleotides against the human epidermal growth factor receptor into cultured KB cells with liposomes conjugated to folate via polyethylene glycol. Proc. Natl. Acad. Sci. 92:3318-3322.
61. Goren, D., Horowitz, A. T., Tzemach, D., Tarshish, M., Zalipsky, S., and Gabizon, A. (2000) Nuclear delivery of doxorubicin via folate-targeted liposomes with bypass of multidrug-resistance efflux pump. Clin. Cancer Res. 6:1949-1957.
62. Qualls, M. M., Thompson, D. H. (2001) Chloroaluminum phtalocyanine tetrasulfonate delivered via acid-labile diplasmenylcholine-folate liposomes: intracellular localization and synergistic photo toxicity. Int J. Cancer 93:384-392.
63. Pan, X. Q., Wang, H., Lee, R. J. (2002) Boron delivery to a murine lung carcinoma using folate receptor-targeted liposomes. Anticancer Res. 22:1629-1633.
64. Reddy, J. A., Abburi, C., Hofland, H., Howard, S. J., Vlahov, I., Wils, P., Leamon, C. P. (2002) Folate-targeted, cationic liposome-mediated gene transfer into disseminated peritoneal tumors. Gene Therapy 9:1542-1550.
65. Zhou, W., Yuan, X., Wilson, A., Yang, L., Mokotoff, M., Pitt, B., Li, S. (2002) Efficient intracellular delivery of oligonucleotides formulated in folate receptor-targeted lipid vesicles. Bioconjug. Chem. 13:1220-1225.
66. Liu, J., Kolar, C., Lawson, T. A., Gmeiner, W. H. (2001) Targeted drug delivery to chemoresistant cells: Folic acid derivatization of FdUMP[10] enhances cytotoxicity toward 5-FU-resistant human colorectal tumor cells. J. Org. Chem. 66:5655-5663.
67. Lu, J. Y., Lowe, D. A., Kennedy, M. D., and Low, P. C. (1999) Folate-targeted enzyme prodrug cancer therapy utilizing penicillin-V amidase and a doxorubicin prodrug. J. Drug Target 7:43-53.
68. Quintana, A., Raczka, E., Piehler, L., Lee, I., Myc. A., Majoros, I., Patri, A. K., Thomas, T., Mule, J., Baker, J. R., Jr. Design and function of a dendrimer-based therapeutic nanodevice targeted to tumor cells through the folate receptor. Pharm. Res. 19:1310-1316.
69. Ward, C. M., Pechar, M., Oupicky, D., Ulbrich, K., Seymour, L. W. (2002) Modification of pLL/DNA complexes with a multivalent hydrophilic polymer permits folate-mediated targeting in vitro and prolonged plasma circulation in vivo. J. Gene Med. 4:536-547.
70. Dauty, E., Remy, J. S., Zuber, G., Behr, J. P. Intracellular delivery of nanometric DNA particles via the folate receptor. Bioconjug. Chem. 13:831-839.
71. Benns, J. M., Maheshwari, A., Furgeson, D. Y., Mahato, R. I., Kim, S. W. (2001) Folate-PEG-folate-graft-polyethylenimine-based gene delivery. J. Drug Target 9:123-139.
72. Westerhof, G. R., Schornagel, J. H., Kathmann, I., et al. (1995) Carrier- and receptor-mediated transport of folate antagonist targeting folate-dependent enzymes: correlates of molecular structure and biological activity. Mol. Pharmacol. 48:459-471.
73. 73a. Jackman, A. L., Theti, D. S., Lornaime, A. S., and Bavetsias, V. B. (2000) Exploitation of the α-isoform of the folate receptor (α-FR) for the selective delivery of antifolate thymidylate synthase (TS) inhibitors. Proc. Am. Assoc. for Cancer Res. 41, Abstract #33. 73b. Bavetsias, V., Marriott, J. H., Melin, C., Kimbell, R., Matusiak, Z. S., Boyle, F. T., Jackman, A. L. (2000) Design and synthesis of cyclopenta[g]quinazoline-based antifolates as inhibitors of thymidylate synthease and potential antitumor agents. J. Med. Chem. 43:1919-1926.
74. Mazzoni, A., Mezzanzanica, D., Jung, G., Wolf, H., Colnaghi, M. I., and Canevari, S. (1996) CD3-CD28 costimulation as a means to avoiding T cell preactivation in bispecific monoclonal antibody-based treatment of ovarian carcinoma. Cancer Res. 56:5443-5449.
75. Mezzanzanica, D., Garrido, M. A., Neblock, D. S., et al. (1991) Human T-lymphocytes targeted against an established human ovarian carcinoma with a bispecific F(ab′)2 antibody prolong host survival in a murine xenograft model. Cancer Res. 51:5716-5721.
76. Canevari, S., Mezzanzanica, D., Mazzoni, A., et al. (1995) Bispecific antibody targeted T cell therapy of ovarian cancer: clinical results and future directions. J. Hematother. 4:423-427.
77. Canevari, S., Mezzanzanica, D., Mazzoni, A., et al. (1997) Approaches to implement bispecific antibody treatment of ovarian carcinoma. Cancer Immunol. Immunother., 45:197-189.
78. Luiten, R. M., Warnaar, S. O., Sanborn, D., et al. (1997) Chimeric bispecific OC/TR monoclonal antibody mediates lysis of tumor cells expressing the folate-binding protein (Mov 18) and displays decreased immunogenicity in patients. J. Immunother. 20:496-504.
79. Melani, C., Figini, M., Nicosia, D., et al. (1998) Targeting of interleukin 2 to human ovarian carcinoma by fusion with a single-chain FR of antifolate receptor antibody. Cancer. Res. 58:4146-4154.
80. Rodolfo, M., Melani, C., Zilocchi, C., et al. (1998) IgG2a induced by interleukin (IL) 12-producing tumor cell vaccines but not IgG1 induced by IL-4 vaccine is associated with the eradication of experimental metastases. Cancer Res. 58:5812-5817.
81. Neglia, F., Orengo, A. M., Cilli, M., et al. (1999) DNA vaccination against the ovarian carcinoma-associated antigen folate receptor alpha (FR alpha) induces cytotoxic T lymphocyte and antibody responses in mice. Cancer Gene Ther. 6:349-357.
82. Rodolfo, M., Zilocchi, C., Cappetti, B., Parmiani, G., Melani, C., and Colombo, M. P. (1999) Cytotoxic T lymphocyte response against non-immunoselected tumor antigens predicts the outcome of gene therapy with IL-12-transduced tumor cell vaccine. Gene Ther. 6:865-872.
83. Peoples, G. E., Anderson, B. W., Lee, T. V., Murray, J. L., Kudelka, A. P., Wharton, J. T., and loannides, C. G. (1999) Vaccine implications of folate binding protein, a novel cytotoxic T lymphocyte-recognized antigen system in epithelial cancers. Clin. Cancer Res. 5:4214-4223.
84. Kim, D. K, Lee, T. V., Castilleja, A., Anderson, B. W., Peoples, G. E., Kudelka, A. P., Murray, J. L., Sittisomwong, T., Wharton, J. T., Kim, J. W., and loannides, CG (1999) Folate binding protein peptide 191-199 presented on dendritic cells can stimulate CTL from ovarian and breast cancer patients. Anticancer Res. 19:2907-2916.
85. Rodolfo, M., Zilocchi, C., Accornero, P., Cappetti, B., Arioli, I. and Colombo, M. P. (1999) IL-4-transduced tumor cell vaccine induces immunoregulatory type 2 CD8 T lymphocytes that cure lung metastases upon adoptive transfer. J. Immunol. 163:1923-1928.
86. Kershaw, M. H., Westwood, J. A., Hwu, P. (2002) Dual-specific T cells combine proliferation and antitumor activity. Nat. Biotechnol. 20:1221-1227.
87. Lu, Y., Low, P. S. (2002) Folate targeting of haptens to cancer cell surfaces mediates immunotherapy of syngenic murine tumors. Cancer Immunol. Immunother. 51:153-162.
1a. Zhang, X., Yan, W., Kelley, K. M. M., Dorn, T., and Ratnam, M. mRNA instability in the nucleus due to a novel open reading frame element is a major determinant of the narrow tissue specificity of folate receptor α. Mol. Cell. Biol. 23: 2202-2212.
2a. Maminta, M. L. D., Molteni, A., Rosen, S. T. Stable expression of the human estrogen receptor in HeLa cells by infection: effect of estrogen on cell proliferation and c-myc expression. Mol. Cell. Endocrinol., 78: 61-69, 1991.
3a. Moggs, J. G., and Orphanides, G. Estrogen receptors: orchestrators of pleiotropic cellular responses. EMBO Rep., 2: 775-781, 2001.
4a. Aronica, S. M., and Katzenellenbogen, B. S. Stimulation of estrogen receptor-mediated transcription and alteration in the phosphorylation state of the rat uterine estrogen receptor by estrogen, cyclic adenosine monophosphate, and insulin-like growth factor-1. Mol. Endocrinol., 7: 743-752, 1993.
5a. McDonnell, D. P. The Molecular Pharmacology of SERMs. Trends Endocrinol. Metab., 10: 301-311,1999.
6a. Parker, M. G. Action of “pure” antiestrogens in inhibiting estrogen receptor action. Breast Cancer Res. Treat., 26: 131-137, 1993.
7a. Dauvois, S., White, R., and Parker, M. G. The antiestrogen ICI-182780 disrupts estrogen receptor nucleocytoplasmic shuttling. J. Cell. Sci., 106: 1377-1388, 1993.
8a. Pink, J. J., and Jordan, V. C. Models of estrogen receptor regulation by estrogens and antiestrogens in breast cancer cell lines. Cancer Res., 56: 2321-2330, 1996.
9a. Klinge, C. M. Estrogen receptor interaction with estrogen response elements. Nucleic Acids Res., 29: 2905-2919, 2001.
10a. Paech, K., Webb, P., Kuiper, G. G., Nilsson, S., Gustafsson, J., Kushner, P. J., and Scanlan, T. S. Differential ligand activation of estrogen receptors ERalpha and ERbeta at AP1 sites. Science, 277: 1508-1510, 1997.
11a. Cerillo, G., Rees, A., Manchanda, N., Reilly, C., Brogan, I., White, A. and Needham, M. The oestrogen receptor regulates NFKappaB and AP-1 activity in a cell-specific manner. J. Steroid Biochem. Mol. Biol., 67: 79-88, 1998.
12a. Webb, P., Nguyen, P., Valentine, C., Lopez, G. N., Kwok, G. R., McInerney, E., Katzenellenbogen, B. S., Enmark, E., Gustafsson, J. Å., Nilsson, S, and Kushner, P. J. The estrogen receptor enhances AP-1 activity by two distinct mechanisms with different requirements for transactivator functions. Mol. Endo., 13: 1672-1685, 1999.
13a. Jakacka, M., Ito, M., Weiss, J., Chien, P. Y., Gehm, B. D., and Jameson, J. L. Estrogen receptor binding to DNA is not required for its activity through the nonclassical AP1 pathway. J. Biol. Chem., 276: 13615-13621, 2001.
14a. An. J., Ribeiro, R. C. J., Webb, P., Gustafsson, J. A., Kushner, P. J., Baxter, J. D. and Leitman, D. C. Estradiol repression of tumor necrosis factor-a transcription requires estrogen receptor activation-2 and is enhanced by coactivators. Proc. Natl. Acad. Sci., 96: 15161-15166, 1999.

15a. Harnish, D. C., Scicchitano, M. S., Adelman, S. J., Lyttle, C. R., and Karathanasis, S. K. The role of CBP in estrogen receptor cross-talk with nuclear factor-kappaB in HepG2 cells. Endocrinology, 141: 3403-3411, 2000.
16a. Yang, N. N., Venugopalan, M., Hardikar, S, and Glasebrook, A. Correction: raloxifene response needs more than an element. Science, 275: 1249, 1997.
17a. Zou, A., Marschke, K. B., Arnold, K. E., Berger, E. M., Fitzgerald, P., Mais, D. E., and Allegretto, E. A. Estrogen receptor beta activates the human retinoic acid receptor alpha-1 promoter in response to tamoxifen and other estrogen receptor antagonists, but not in response to estrogen. Mol. Endocrinol., 13: 418-430, 1999.
18a. Safe, S. Transcriptional activation of genes by 17 beta-estradiol through estrogen receptor-Sp1 interactions. Vitam. Horm., 62: 231-252, 2001.
19a. Elgort, M. G., Zou, A., Marschke, K. B., and Allegretto, E. A. Estrogen and estrogen receptor antagonists stimulate transcription from the human retinoic acid receptor-alpha 1 promoter via a novel sequence. Mol. Endocrinol., 10: 477-487, 1996.
20a. Stoner, M., Wang, F., Wormke, M., Nguyen, T., Samudio, I., Vyhlidal, C., Marme, D., Finkenzeller, G., and Safe, S. Inhibition of vascular endothelial growth factor expression in HEC1A endometrial cancer cells through interactions of estrogen receptor alpha and Sp3 proteins. J. Biol. Chem., 275: 22769-22779, 2000.
21a. Blobel, G. A., Sieff, C. A., and Orkin, S. H. Ligand-dependent repression of the erythroid transcription factor GATA-1 by the estrogen receptor. Mol. Cell. Biol., 15: 3147-3153, 1995.
22a. Kuiper, G. G., Enmark, E., Pelto-Huikko, M., Nilsson, S., and Gustafsson, J. A. Cloning of a novel receptor expressed in rat prostate and ovary. Proc Natl. Acad. Sci. USA, 93: 5925-5930, 1996.
23a. Pujol, P., Rey, J. M., Nirde, P., Roger, P., Gastaldi, M., Laffargue, F., Rochefort, H., and Maudelonde, T. Differential expression of estrogen receptor-alpha and -beta messenger RNAs as a potential marker of ovarian carcinogenesis. Cancer Res., 58: 5367-5373, 1998.
24a. Cowley, S. M., Hoare, S., Mosselman, S., and Parker, M. G. Estrogen receptors alpha and beta form heterodimers on DNA. J. Biol. Chem., 272: 19858-19862, 1997.
25a. Tremblay, G. B., Tremblay, A., Labrie, F., and Giguere, V. Dominant activity of activation function 1 (AF-1) and differential stoichiometric requirements for AF-1 and -2 in the estrogen receptor alpha-beta heterodimeric complex. Mol. Cell. Biol., 19: 1919-1927, 1999.
26a. Pettersson, K., and Gustafsson, J. A. Role of estrogen receptor beta in estrogen action. Annu. Rev. Physiol., 63: 165-192, 2001.
27a. Saville, B., Wormke, M., Wang, F., Nguyen, T., Enmark, E., Kuiper, G., Gustafsson, J. A., and Safe, S. Ligand-, cell-, and estrogen receptor subtype (alpha/beta)-dependent activation at GC-rich (Sp1) promoter elements. J. Biol. Chem., 275: 5379-5387, 2000.
28a. Jordan, V. C. Targeted Antiestrogens to Prevent Breast Cancer. Trends Endocrinol. Metab., 10: 312-317, 1999.

I claim:

1. A method of diagnosing and treating a patient with or suspected of having at least one solid tumor that produces folate receptor a comprising administering a biologically effective amount of at least one folate receptor α inducer, the at least one folate receptor α inducer comprising at least one steroid receptor agent, optionally together with one or more histone deacetylase inhibitors, wherein the at least one folate receptor α inducer increases levels of folate receptor α on plasma membranes of tumor cells as well as folate receptor α in at least one type of body fluid.

2. The method of claim 1, wherein the body fluid includes at least one of serum, ascites and cerebrospinal fluid.

3. The method of claim 1, wherein a diagnostic assay is performed using samples of said body fluid.

4. The method of claim 1, wherein diagnostic tumor imaging is performed using at least one imaging agent directed at folate receptor α.

5. The method of claim 1, wherein the patient is treated by administering a therapeutic agent that targets folate receptor α expressed by tumor cells.

6. The method of claim 1, wherein the steroid receptor comprises at least one of estrogen receptor (ER), progesterone receptor (PR), androgen receptor (AR) or glucocorticoid receptor (GR).

7. The method of claim 1, wherein the folate receptor α inducer comprises at least one or more agonist or antagonist agent of at least one steroid receptor.

8. The method of claim 7, wherein the steroid receptor agent comprises at least one of tamoxifen, progestin, androgens and dexamethasone and optionally, any other agent that acts on estrogen receptor (ER), progesterone receptor (PR), androgen receptor (AR) or glucocorticoid receptor (GR).

9. The method of claim 8, wherein the diagnosis and/or treatment of a patient comprises administering at least one histone deacetylase inhibitor to the patient together with one or more steroid receptor agonists or antagonists wherein the histone deactylase inhibitor includes at least one of trichostatin A (TSA), suberoylanilide hydroxamic acid (SAHA), depsipeptide, valproic acid, butyrates, or other HDAC inhibitor.

10. The method of claims 1 through 9, wherein a composition for assaying folate receptors in serum and ascites comprises at least one of fluorescent conjugates of folate or antibodies specific for the folate receptor is used.

11. The method of claims 1 through 9, wherein a composition for therapeutic targeting of the folate receptor comprising at least one of folate-coated liposomal drugs, folate conjugated nanoparticle drug delivery systems, antifolate drugs, folate conjugated radiopharmaceuticals or cytotoxics, or folate receptor-targeted immunotherapeutics is used.

12. The method of claim 6, wherein the steroid receptor comprises the glucocorticoid receptor (GR).

13. The method of claim 12, wherein the steroid receptor agent comprises dexamethasone or other GR agonist.

14. A method for diagnosing and/or therapeutically treating a patient comprising activating the FR-α gene in the patient using at least one of an agonist or antagonist of estrogen receptor (ER), progesterone receptor (PR), glucocorticoid receptor (GR) or androgen receptor (AR).

15. The method of claim 14, wherein the agonist or antagonist comprises at least one of tamoxifen, progestin, androgens and dexamethasone and optionally, any other steroid receptor agent that acts on estrogen receptor (ER), progesterone receptor (PR), androgen receptor (AR) or glucocorticoid receptor (GR).

16. The method of claim 15, wherein the steroid receptor comprises glucocorticoid receptor (GR) and the steroid receptor agent comprises dexamethasone or other GR agonist.

17. The method of claim 16, further comprising administering at least one histone deacetylase inhibitor.

18. The method of claim 15, wherein the steroid receptor comprises estrogen receptor (ER) and the steroid receptor agent comprises tamoxifen.

19. The method of claim 18, further comprising administering at least one histone deacetylase inhibitor.

20. The method of claim 15, wherein the steroid receptor comprises androgen receptor (AR) and the steroid receptor agent comprises at least one of testosterone, dihydroxytestosterone or other AR agonist.

21. The method of claim 20, further comprising administering at least one histone deacetylase inhibitor.

22. The method of claim 15, wherein the steroid receptor comprises progesterone receptor (PR) and the steroid receptor agent comprises progestin or other PR agonist.

23. The method of claim 20, further comprising administering at least one histone deacetylase inhibitor.

24. A method for substantially increasing folate receptor (FR) type a expression in a tumor comprising using at least one steroid receptor agent, individually or in combination, to substantially increase folate receptor (FR) type α expression in the tumor in a manner selective to FR-α-positive tissues.

25. The method of claim 24, wherein the steroid receptor agent comprises at least one agonist or antagonist including tamoxifen, progestin, androgens and dexamethasone and optionally, any other steroid receptor agent that acts on estrogen receptor (ER), progesterone receptor (PR), androgen receptor (AR) or glucocorticoid receptor (GR).

26. The method of claim 25, wherein the steroid receptor comprises glucocorticoid receptor (GR) and the steroid receptor agent comprises dexamethasone.

27. The method of claim 26, further comprising administering at least one histone deacetylase inhibitor.

28. The method of claim 25, wherein the steroid receptor comprises estrogen receptor (ER) and the steroid receptor agent comprises tamoxifen.

29. The method of claim 28, further comprising administering at least one histone deacetylase inhibitor.

30. The method of claim 25, wherein the steroid receptor comprises androgen receptor (AR) and the steroid receptor agent comprises at least one of testosterone or dihydroxytestosterone or other AR agonist.

31. The method of claim 30, further comprising administering at least one histone deacetylase inhibitor.

32. The method of claim 25, wherein the steroid receptor comprises progesterone receptor (PR) and the steroid receptor agent comprises progestin or other PR agonist.

33. The method of claim 32, further comprising administering at least one histone deacetylase inhibitor.

34. A method of manipulation of tumor tissues comprising using at least one steroid receptor agent to enhance sensitivity of whole body imaging and therapeutic efficacy of FR-targeted agents in the treatment of cancer and to increase the level of FR-α in circulation.

35. The method of claim 34, wherein the steroid receptor agent comprises at least one agonist or antagonist including tamoxifen, progestin, androgens and dexamethasone and optionally, any other steroid receptor agent that acts on estrogen receptor (ER), progesterone receptor (PR), androgen receptor (AR) or glucocorticoid receptor (GR).

36. The method of claim 35, wherein the steroid receptor comprises glucocorticoid receptor (GR) and the steroid receptor agent comprises dexamethasone or other GR agonist.

37. The method of claim 36, further comprising administering at least one histone deacetylase inhibitor.

38. The method of claim 35, wherein the steroid receptor comprises estrogen receptor (ER) and the steroid receptor agent comprises tamoxifen or other ER agonist or antagonist.

39. The method of claim 38, further comprising administering at least one histone deacetylase inhibitor.

40. The method of claim 35, wherein the steroid receptor comprises androgen receptor (AR) and the steroid receptor agent comprises at least one of testosterone or dihydroxytestosterone or other AR agonist.

41. The method of claim 40, further comprising administering at least one histone deacetylase inhibitor.

42. The method of claim 35, wherein the steroid receptor comprises progesterone receptor (PR) and the steroid receptor agent comprises progestin or other PR agonist.

43. The method of claim 42, further comprising administering at least one histone deacetylase inhibitor.

44. A method of enhancing sensitivity of whole body imaging and therapeutic efficacy of FR-targeted agents in the treatment of cancer comprising manipulating tumor tissues by using steroid receptor agents that bind to folate receptor α.

45. The method of claim 44, wherein the steroid receptor agent comprises at least one agonist or antagonist including tamoxifen, progestin, androgens and dexamethasone and optionally, any other steroid receptor agent that acts on estrogen receptor (ER), progesterone receptor (PR), androgen receptor (AR) or glucocorticoid receptor (GR).

46. The method of claim 45, wherein the steroid receptor comprises glucocorticoid receptor (GR) and the steroid receptor agent comprises dexamethasone or other GR agonist.

47. The method of claim 46, further comprising administering at least one histone deacetylase inhibitor.

48. The method of claim 45, wherein the steroid receptor comprises estrogen receptor (ER) and the steroid receptor agent comprises tamoxifen or other ER agonist or antagonist.

49. The method of claim 48, further comprising administering at least one histone deacetylase inhibitor.

50. The method of claim 45, wherein the steroid receptor comprises androgen receptor (AR) and the steroid receptor agent comprises at least one of testosterone or dihydroxytestosterone or other AR agonist.

51. The method of claim 50, further comprising administering at least one histone deacetylase inhibitor.

52. The method of claim 45, wherein the steroid receptor comprises progesterone receptor (PR) and the steroid receptor agent comprises progestin or other PR agonist.

53. The method of claim 52, further comprising administering at least one histone deacetylase inhibitor.

54. A method for increasing levels of FR-α in a patient's circulation comprising manipulating tumor tissues by using steroid receptor agents that bind to folate receptor α.

55. The method of claim 54, wherein the steroid receptor agent comprises at least one agonist or antagonist including tamoxifen, progestin, androgens and dexamethasone and optionally, any other steroid receptor agent that acts on estrogen receptor (ER), progesterone receptor (PR), androgen receptor (AR) or glucocorticoid receptor (GR).

56. The method of claim 55, wherein the steroid receptor comprises glucocorticoid receptor (GR) and the steroid receptor agent comprises dexamethasone or other GR agonist.

57. The method of claim 56, further comprising administering at least one histone deacetylase inhibitor.

58. The method of claim 55, wherein the steroid receptor comprises estrogen receptor (ER) and the steroid receptor agent comprises tamoxifen or other ER agonist or antagonist.

59. The method of claim 58, further comprising administering at least one histone deacetylase inhibitor.

60. The method of claim 55, wherein the steroid receptor comprises androgen receptor (AR) and the steroid receptor agent comprises at least one of testosterone or dihydroxytestosterone or other AR agonist.

61. The method of claim 60, further comprising administering at least one histone deacetylase inhibitor.

62. The method of claim 55, wherein the steroid receptor comprises progesterone receptor (PR) and the steroid receptor agent comprises progestin or other PR agonist.

63. The method of claim 62, further comprising administering at least one histone deacetylase inhibitor.

62. A serum marker for diagnostic screening and monitoring of cancers during and after treatment comprising the composition of claim 9.


SEQUENCE LISTING (18 sequences)

SEQ ID NO: 1 - DNA, 223 bp, Homo sapiens
gtgaccacctggagaaggcaatgaggctcaagccagggaggggtggtgtctaatcctacctttcattggatctgggaaaactgagggagatgggggcagggctctatctgccccaggcttccgtccaggccccaccctcctggagccctgcacacaacttaaggccccacctccgcattccttggtgccactgaccacagctctttcttcagggacagacatg

SEQ ID NO: 2 - DNA, 12 bp, Homo sapiens
tgaggctcaagc

SEQ ID NO: 3 - DNA, 13 bp, Homo sapiens
gggaggggtggtg

SEQ ID NO: 4 - DNA, 22 bp, Homo sapiens
ctgagggagatgggggcagggc

SEQ ID NO: 5 - DNA, 11 bp, Homo sapiens
ccccaccctcc

SEQ ID NO: 6 - DNA, 2723 bp, Homo sapiens
ttggaaactgatgagattagctcaaaggatcctggcagctcaggctgcaagatttttttcagacctcagtgtttgggaaaaaattgggtaggtggagcttagggactggccttaggcctgcactgttaattcaccccctcccactaccccatggaggcctggctggtgctcacatacaataattaactgctgagtggccttcgcccaatcccaggctccactcctgggctccattcccactccctgcctgtctcctaggccactaaaccacagctgtcccctggaataaggcaagggggagtgtagagcagagcagaagcctgagccagacggagagccacctcctctcccaggtatgtgacactccccatcccccttcagaggccacacaccctatggcattcccaccatgtgttaaggattttctgaactggaagggccctctgtttgcctgaaggccagagaatcttgaagtggagactgaggcccagaccagagtgtggcctgctcaagattaaacgacaagttagtgttcatccccctgaactagtacctgggctctagcccttcagtccagagctgagttctcagctcttctagtctggggccccaaggttgggtgtgggggtcatgattgttggtggggaggggtcacagctggactaagacctgaaggtgagactaggcaggtgggaaaggagcttgcagagtgatgctgctcaaaaggacaggaagagagcctggcttcagaagcagccacagcaagagagactactgactgaacaggtgggctccactgggggctccggaaaggattttctcagcccccatccccagcactgtgtgttggccgcacccatgagagcctcagcactctgaaggtgcagggggcaaaggccaaaagagctctggcctgaacttgggtggtccctactgtgtgacttggggcatggccctcatctgtgctgaaatgattccacaaagattaaactggctatcatttgttgatttcccccttcttacatttaatccttgcaggagaaagctaagcctcaagatagtttgcttctctttcccccaaggccaaggagaaggtggagtgagggctggggtcgggacaggttgaacgggaaccctgtgctctaaacagttagggtttgttcccgcaggaactgaacccaaaggatcacctggtattccctgagagtacagatttctccggcgtggccctcaaggttagtgagtgagcaggtccacaggggcatgattggatcctggaatgaatgaatcaaccatgagagagtgaatgaacactggaatcaatagagtagcagagtaatggattgtggagcaggaaagagagctgctgggtgggaattcaattccaggcttatatgagccctgctgtgcagtcggcctggagacagcccagctcaggccctgcctagacccctgtcaaggaggccctgtcaagaggagaggaggggcagcacgggggcaaggcaagcttgtgagcgggaaaggcatgtccactttagcgactggtatgtggaagatgagttagaggagacagatggagagaagtcataggaaataaattctgagcattttaggagggcccagacacctggtgtccagtggagtgaaggaaacagtcgcctcccaaaattcagtgtctgaggtcaaaggattgaagttctgtgatgaccaaggagaagccagctctgtggtagggggcacaggagctccccaaggccccagggctgtccagctggctgtcccctgccagcacccatgtcctgtgaccccaccccaccaagatcccatggtttccgggaagggcctactaaactagcttgagtgatgaggctagaaaggggctgggaccaaggtttaaaaagcaaaacaaactaacaaaaaccacactgcagcccccccaactaaaacatttttataaactttttttttttttttgagatggagtctcgctctgtcacccaggctagagtgcaatggcacaatcttggctcactgtaacctccacctcctggattcaagtgattctcctgcctcagcctcccacgtagctgggactacaggcacacgacaccgcacccagctcattttgtatttttagtagagacagggtttcactatgttggccaggctggtctcaaacttctgacctcaggtgatccacccacctcagccttccaaagtgctgggattacaggcatgagccaccgcgcccagcccatttttgtaaacttttacaatgaagtaatttggtgtcaaaatctgacctgaaaattaatgtgagtttatgtatagttttaatttatcccactagtgtaactgtttcaccccagaatatacacttgattattgggtatatgaaaaaaatattttctttgaatcacctttgatgaaatcctaaaaaattttaaccctgaaacatttgaataaggcattgtggacctatggcaaactcctggctatttctgcattttgcccaaatccatccttgaattatatcacctgaacctcgtgaccacctggagaaggcaatgaggctcaagccagggaggggtggtgtctaatcctacctttcattggatctgggaaaactgagggagatgggggcagggctctatctgccccaggcttccgtccaggccccaccctcctggagccctgcacacaacttaaggccccacctccgc

SEQ ID NO: 7 - DNA, 105 bp, Homo sapiens
gggaggggtggtgtctaatcctacctttcattggatctgggaaaactgagggagatgggggcagggctctatctgccccaggcttccgtccaggccccaccctcc

SEQ ID NO: 8 - DNA, 47 bp, Homo sapiens
gcattccttggtgccactgaccacagctctttcttcagggacagaca

SEQ ID NO: 9 - DNA, 22 bp, Homo sapiens
gtcagcatatgtagtcccgccc

SEQ ID NO: 10 - DNA, 21 bp, Homo sapiens
aaacttaagcagcgatggggc

SEQ ID NO: 11 - DNA, 21 bp, Homo sapiens
attctccgcggcatcgctgac

SEQ ID NO: 12 - DNA, 22 bp, Homo sapiens
cactgcatacgacgattctgtg

SEQ ID NO: 13 - DNA, 21 bp, Homo sapiens
attcgatcggggcggggcgag

SEQ ID NO: 14 - DNA, 20 bp, Homo sapiens
gtcaggtcacagtgacctga

SEQ ID NO: 15 - DNA, 1095 bp, Homo sapiens
ttggaaactgatgagattagctcaaaggatcctggcagctcaggctgcaagatttttttcagacctcagtgtttgggaaaaaattgggtaggtggagcttagggactggccttaggcctgcactgttaattcaccccctcccactaccccatggaggcctggctggtgctcacatacaataattaactgctgagtggccttcgcccaatcccaggctccactcctgggctccattcccactccctgcctgtctcctaggccactaaaccacagctgtcccctggaataaggcaagggggagtgtagagcagagcagaagcctgagccagacggagagccacctcctctcccaggtatgtgacactccccatcccccttcagaggccacacaccctatggcattcccaccatgtgttaaggattttctgaactggaagggccctctgtttgcctgaaggccagagaatcttgaagtggagactgaggcccagaccagagtgtggcctgctcaagattaaacgacaagttagtgttcatccccctgaactagtacctgggctctagcccttcagtccagagctgagttctcagctcttctagtctggggccccaaggttgggtgtgggggtcatgattgttggtggggaggggtcacagctggactaagacctgaaggtgagactaggcaggtgggaaaggagcttgcagagtgatgctgctcaaaaggacaggaagagagcctggcttcagaagcagccacagcaagagagactactgactgaacaggtgggctccactgggggctccggaaaggattttctcagcccccatccccagcactgtgtgttggccgcacccatgagagcctcagcactctgaaggtgcagggggcaaaggccaaaagagctctggcctgaacttgggtggtccctactgtgtgacttggggcatggccctcatctgtgctgaaatgattccacaaagattaaactggctatcatttgttgatttcccccttcttacatttaatccttgcaggagaaagctaagcctcaagatagtttgcttctctttcccccaaggccaaggagaagg

SEQ ID NO: 16 - DNA, 2723 bp, Homo sapiens; identical to SEQ ID NO: 6

SEQ ID NO: 17 - DNA, 41 bp, Homo sapiens
ggagatgggggcagggctctatctgccccaggcttccgtcc

SEQ ID NO: 18 - DNA, 100 bp, Homo sapiens
gatgaggctagaaaggggctgggaccaaggtttaaaaagcaaaacaaactaacaaaaaccacactgcagcccccccaactaaaacatttttataaacttt